(19) United States
(12) Patent Application Publication
(10) Pub. No.: US 2022/0163789 A1
(43) Pub. Date: May 26, 2022

(54) CONDENSING OBJECTIVE OPTICAL SYSTEM AND PHOTOACOUSTIC DEVICE

(71) Applicant: TAMRON CO., LTD., Saitama (JP)

(72) Inventors: Shingo FUSE, Saitama (JP); Shunya FUKUI, Saitama (JP)

(73) Assignee: TAMRON CO., LTD., Saitama (JP)

(21) Appl. No.: 17/499,419

(22) Filed: Oct. 12, 2021

(30) Foreign Application Priority Data
Nov. 26, 2020 (JP) 2020-196356

(51) Int. Cl. G02B 26/08; G02B 9/10; A61B 5/00

(52) U.S. Cl. CPC G02B 26/0875; A61B 5/0095; G02B 9/10

(57) ABSTRACT

A condensing objective optical system includes a first lens group having negative refractive power, a second lens group being a focus group having positive refractive power, and a photoacoustic element disposed closest to an object in this order. The photoacoustic element reflects an optical wave from the second lens group toward an object to transmit a photoacoustic wave from the object side. The condensing objective optical system satisfies a specific conditional expression that defines the conditions of the included lens and the condition regarding focusing.

### CROSS-REFERENCE TO RELATED APPLICATIONS

This application is based on and claims the benefit of priority from Japanese Patent Application No. 2020-196356, filed on Nov. 26, 2020, the entire contents of which are incorporated herein by reference.

### BACKGROUND OF THE INVENTION

### Technical Field

The present invention relates to a condensing objective optical system and a photoacoustic device.

### Related Art

In the technical field of imaging the inside of a living body, examination of an imaging technique of the inside of a living body using photoacoustics is in progress. In an imaging technique using photoacoustics, a tissue in a living body is irradiated with a laser to generate an ultrasonic wave.

On the other hand, various studies have been heretofore made on an optical system used in an imaging technique using photoacoustics. In the optical system, a configuration having a large hole diameter and field of view, and a long operation distance, a configuration having a high numerical aperture on the image side and performing focusing in the entire optical system, a configuration performing focusing using an expander unit, and the like are known (see, for example, JP 2016-503514 A, JP 2005-258336 A, and JP 2015-205346 A).

One of the main applications of focusing technology is application of laser processing. In this case, the environment from the optical element to the target object is usually an air atmosphere. On the other hand, in a case where photoacoustics are used to observe the inside of a living body, the environment from the optical element to the target object is a living tissue such as blood and fat. In this case, the refractive index n of a target environment is usually larger than one. Note that the “target object” is an object to be imaged in the above-described imaging technique, and is an object that generates a photoacoustic wave by being irradiated with an optical wave.

Here, when the environment is an air atmosphere, the relationship between the distance of focusing by the focus lens in the optical system and the distance from the optical system to the target object is 1:1. In the observation of the inside of the living body by photoacoustics, since the refractive index of the living tissue is larger than one, the collection efficiency may be significantly reduced when focusing is performed in a 1:1 relationship as in the case of the air atmosphere.

As described above, the conventional technique assumes an air atmosphere as an environment from the optical system to the target object. Therefore, the conventional technique has a problem that, in observation of a target object having a refractive index larger than that of air, such as observation of the inside of a living body, light condensing performance is deteriorated during focusing.

An object of an aspect of the present invention is to realize a photoacoustic optical system that can also be used for observation of the inside of a living body.

### SUMMARY OF THE INVENTION

In order to solve the above problem, a condensing objective optical system according to an aspect of the present invention includes a first lens group having negative refractive power, a second lens group having positive refractive power and being movable along an optical axis so as to change a distance between the second lens group and an adjacent lens group, and a photoacoustic element disposed closest to an object, reflecting an optical wave incident from the second lens group toward the object, and transmitting a photoacoustic wave emitted by the object that has absorbed the optical wave, in this order, wherein the condensing objective optical system includes at least one lens that satisfies the following expressions (1) and (2), and satisfies the following expression (3):

νd>64 (1)

0.294≤f/fν MAX<2.140 (2)

1.000<ΔZ/Lf≤2.386 (3)

where

νd is an Abbe number with respect to d Line, f is a focal distance of the condensing objective optical system when the object distance is longest,

fνMAX is a focal distance of the lens the νd of which is maximum,

ΔZ is an amount of change in a distance on an optical axis from a surface, of the photoacoustic element, on object side to an imaging position at the object side in the condensing objective optical system, and

Lf is a movement distance of the second lens group.

In addition, in order to solve the above-described problem, a photoacoustic device according to an aspect of the present invention includes the above-described condensing objective optical system, and detects a photoacoustic wave emitted by the object that has absorbed an optical wave emitted from the condensing objective optical system.

According to an aspect of the present invention, it is possible to realize a photoacoustic optical system that can also be used for observation of the inside of a living body.

### BRIEF DESCRIPTION OF THE DRAWINGS

FIG. 1 is a block diagram schematically illustrating a configuration of a photoacoustic device according to an embodiment of the present invention;

FIG. 2 is a diagram schematically illustrating an optical configuration according to an object distance of a condensing objective optical system in Example 1 of the present invention;

FIG. 3 is a diagram illustrating a graph of spherical aberration and a graph of longitudinal chromatic aberration at an operation distance 1 in Example 1 of the present invention;

FIG. 4 is a diagram illustrating a graph of spherical aberration and a graph of longitudinal chromatic aberration at an operation distance 2 in Example 1 of the present invention;

FIG. 5 is a diagram illustrating a graph of spherical aberration and a graph of longitudinal chromatic aberration at an operation distance 3 in Example 1 of the present invention;

FIG. 6 is a diagram schematically illustrating an optical configuration according to an object distance of a condensing objective optical system in Example 2 of the present invention;

FIG. 7 is a diagram illustrating a graph of spherical aberration and a graph of longitudinal chromatic aberration at an operation distance 1 in Example 2 of the present invention;

FIG. 8 is a diagram illustrating a graph of spherical aberration and a graph of longitudinal chromatic aberration at an operation distance 2 in Example 2 of the present invention;

FIG. 9 is a diagram illustrating a graph of spherical aberration and a graph of longitudinal chromatic aberration at an operation distance 3 in Example 2 of the present invention;

FIG. 10 is a diagram schematically illustrating an optical configuration according to an object distance of a condensing objective optical system in Example 3 of the present invention;

FIG. 11 is a diagram illustrating a graph of spherical aberration and a graph of longitudinal chromatic aberration at an operation distance 1 in Example 3 of the present invention;

FIG. 12 is a diagram illustrating a graph of spherical aberration and a graph of longitudinal chromatic aberration at an operation distance 2 in Example 3 of the present invention;

FIG. 13 is a diagram illustrating a graph of spherical aberration and a graph of longitudinal chromatic aberration at an operation distance 3 in Example 3 of the present invention;

FIG. 14 is a diagram schematically illustrating an optical configuration according to an object distance of a condensing objective optical system in Example 4 of the present invention;

FIG. 15 is a diagram illustrating a graph of spherical aberration and a graph of longitudinal chromatic aberration at an operation distance 1 in Example 4 of the present invention;

FIG. 16 is a diagram illustrating a graph of spherical aberration and a graph of longitudinal chromatic aberration at an operation distance 2 in Example 4 of the present invention;

FIG. 17 is a diagram illustrating a graph of spherical aberration and a graph of longitudinal chromatic aberration at an operation distance 3 in Example 4 of the present invention;

FIG. 18 is a diagram schematically illustrating an optical configuration according to an object distance of a condensing objective optical system in Example 5 of the present invention;

FIG. 19 is a diagram illustrating a graph of spherical aberration and a graph of longitudinal chromatic aberration at an operation distance 1 in Example 5 of the present invention;

FIG. 20 is a diagram illustrating a graph of spherical aberration and a graph of longitudinal chromatic aberration at an operation distance 2 in Example 5 of the present invention;

FIG. 21 is a diagram illustrating a graph of spherical aberration and a graph of longitudinal chromatic aberration at an operation distance 3 in Example 5 of the present invention;

FIG. 22 is a diagram schematically illustrating an optical configuration according to an object distance of a condensing objective optical system in Example 6 of the present invention;

FIG. 23 is a diagram illustrating a graph of spherical aberration and a graph of longitudinal chromatic aberration at an operation distance 1 in Example 6 of the present invention;

FIG. 24 is a diagram illustrating a graph of spherical aberration and a graph of longitudinal chromatic aberration at an operation distance 2 in Example 6 of the present invention;

FIG. 25 is a diagram illustrating a graph of spherical aberration and a graph of longitudinal chromatic aberration at an operation distance 3 in Example 6 of the present invention;

FIG. 26 is a diagram schematically illustrating an optical configuration according to an object distance of a condensing objective optical system in Example 7 of the present invention;

FIG. 27 is a diagram illustrating a graph of spherical aberration and a graph of longitudinal chromatic aberration at an operation distance 1 in Example 7 of the present invention;

FIG. 28 is a diagram illustrating a graph of spherical aberration and a graph showing an axial chrominance at an operation distance 2 in Example 7 of the present invention;

FIG. 29 is a diagram illustrating a graph of spherical aberration and a graph showing an axial chrominance at an operation distance 3 in Example 7 of the present invention;

FIG. 30 is a diagram schematically illustrating an optical configuration according to an object distance of a condensing objective optical system in Example 8 of the present invention;

FIG. 31 is a diagram illustrating a graph of spherical aberration and a graph of longitudinal chromatic aberration at an operation distance 1 in Example 8 of the present invention;

FIG. 32 is a diagram illustrating a graph of spherical aberration and a graph of longitudinal chromatic aberration at an operation distance 2 in Example 8 of the present invention;

FIG. 33 is a diagram illustrating a graph of spherical aberration and a graph of longitudinal chromatic aberration at an operation distance 3 in Example 8 of the present invention;

FIG. 34 is a diagram schematically illustrating an optical configuration according to an object distance of a condensing objective optical system in Example 9 of the present invention;

FIG. 35 is a diagram illustrating a graph of spherical aberration and a graph of longitudinal chromatic aberration at an operation distance 1 in Example 9 of the present invention;

FIG. 36 is a diagram illustrating a graph of spherical aberration and a graph of longitudinal chromatic aberration at an operation distance 2 in Example 9 of the present invention;

FIG. 37 is a diagram illustrating a graph of spherical aberration and a graph of longitudinal chromatic aberration at an operation distance 3 in Example 9 of the present invention;

FIG. 38 is a diagram schematically illustrating an optical configuration according to an object distance of a condensing objective optical system in Example 10 of the present invention;

FIG. 39 is a diagram illustrating a graph of spherical aberration and a graph of longitudinal chromatic aberration at an operation distance 1 in Example 10 of the present invention;

FIG. 40 is a diagram illustrating a graph of spherical aberration and a graph of longitudinal chromatic aberration at an operation distance 2 in Example 10 of the present invention;

FIG. 41 is a diagram illustrating a graph of spherical aberration and a graph of longitudinal chromatic aberration at an operation distance 3 in Example 10 of the present invention;

FIG. 42 is a diagram schematically illustrating an optical configuration according to an object distance of a condensing objective optical system in Example 11 of the present invention;

FIG. 43 is a diagram illustrating a graph of spherical aberration and a graph of longitudinal chromatic aberration at an operation distance 1 in Example 11 of the present invention;

FIG. 44 is a diagram illustrating a graph of spherical aberration and a graph of longitudinal chromatic aberration at an operation distance 2 in Example 11 of the present invention;

FIG. 45 is a diagram illustrating a graph of spherical aberration and a graph of longitudinal chromatic aberration at an operation distance 3 in Example 11 of the present invention;

FIG. 46 is a diagram schematically illustrating an optical configuration according to an object distance of a condensing objective optical system in Example 12 of the present invention;

FIG. 47 is a diagram illustrating a graph of spherical aberration and a graph of longitudinal chromatic aberration at an operation distance 1 in Example 12 of the present invention;

FIG. 48 is a diagram illustrating a graph of spherical aberration and a graph of longitudinal chromatic aberration at an operation distance 2 in Example 12 of the present invention;

FIG. 49 is a diagram illustrating a graph of spherical aberration and a graph of longitudinal chromatic aberration at an operation distance 3 in Example 12 of the present invention;

FIG. 50 is a diagram schematically illustrating an optical configuration according to an object distance of a condensing objective optical system in Example 13 of the present invention;

FIG. 51 is a diagram illustrating a graph of spherical aberration and a graph of longitudinal chromatic aberration at an operation distance 1 in Example 13 of the present invention;

FIG. 52 is a diagram illustrating a graph of spherical aberration and a graph of longitudinal chromatic aberration at an operation distance 2 in Example 13 of the present invention;

FIG. 53 is a diagram illustrating a graph of spherical aberration and a graph of longitudinal chromatic aberration at an operation distance 3 in Example 13 of the present invention;

FIG. 54 is a diagram schematically illustrating an optical configuration according to an object distance of a condensing objective optical system in Example 14 of the present invention;

FIG. 55 is a diagram illustrating a graph of spherical aberration and a graph of longitudinal chromatic aberration at an operation distance 1 in Example 14 of the present invention;

FIG. 56 is a diagram illustrating a graph of spherical aberration and a graph of longitudinal chromatic aberration at an operation distance 2 in Example 14 of the present invention; and

FIG. 57 is a diagram illustrating a graph of spherical aberration and a graph of longitudinal chromatic aberration at an operation distance 3 in Example 14 of the present invention.

### DESCRIPTION OF THE EMBODIMENTS

1. Condensing Objective Optical System

1-1. Optical Configuration

A condensing objective optical system according to an embodiment of the present invention is an instrument that condenses an optical wave output from a light source, irradiates an object with the optical wave, and detects a photoacoustic wave generated by the irradiation. The condensing objective optical system includes, in order from the light source side, a first lens group having negative refractive power, a second lens group having positive refractive power and being movable along the optical axis so as to change a distance between the second lens group and an adjacent lens group, and a photoacoustic element disposed closest to the object, reflecting an optical wave incident from the second lens group toward the object, and transmitting a photoacoustic wave emitted by the object that absorbed the optical wave. The condensing objective optical system can exhibit sufficient light condensing performance even for a medium having a larger refractive index other than air, and can also be used for observation of a target object covered with such a medium. Note that the “medium” is a portion through which an optical wave from the photoacoustic element to the target object passes.

In the embodiments of the present invention, the “refractive index” means an absolute refractive index unless otherwise specified.

In the present embodiment, the “lens group” means an aggregate of one or a plurality of lens components. The lens group moves while maintaining a relative positional relationship between lens components in the same lens group. In the present embodiment, the first lens group is fixed, and the second lens group is movable along the optical axis. In the embodiment of the present invention, focusing is performed by moving the second lens group along the optical axis.

The lens component is not limited, and may be a lens or a cemented lens. The cemented lens is a lens component in which a plurality of lenses is integrated without an air interval. The lens may be a single lens or a composite lens. The single lens is a lens made of one glass material. The composite lens is a lens in which one single lens and a resin are integrated without an air interval.

Note that the cemented lens is counted as one lens component, and is counted as two lenses. Both the single lens and the composite lens are counted as one lens component, or as one lens.

The condensing objective optical system in the present embodiment may be configured based on a specific medium having a refractive index higher than that of air. For example, it is configured to exhibit sufficiently high light condensing performance even for a medium covering a target object. For example, the condensing objective optical system can be configured by appropriately adjusting the optical performance of the first lens group, the second lens group, and the photoacoustic element, and the configuration of the lens in each lens group so as to satisfy optical conditions described later.

In addition, the condensing objective optical system may further include a lens group other than the above-described lens group or may further include another optical element within a range in which the effect of the present embodiment can be obtained. Examples of other optical elements include a stop, an anti-vibration lens, a filter, a dummy glass, a cover glass, a bandpass filter, and an ND filter (neutral density filter). The “stop” refers to a stop that defines the diameter of the pencil of light in the condensing objective optical system, that is, a stop that defines the F value of the condensing objective optical system. The stop is preferably disposed between the first lens group and the light source, that is, disposed opposite to the photoacoustic element with respect to the first lens group in the direction along the optical axis of the condensing objective optical system from the viewpoint of sufficiently making the collimated optical wave incident on the condensing objective optical system without optical loss.

(1) First Lens Group

The first lens group is a lens group disposed closest to the light source in the condensing objective optical system, and has negative refractive power. The first lens group may be composed of one or a plurality of lens components, and may have negative refractive power as a whole. It is preferable from the viewpoint of reducing an angle of the ray incident on the second lens group and suppressing the aberration at the condensing position that the first lens group includes a lens component having positive refractive power. In addition, it is preferable from the viewpoint of suppressing aberration at the condensing position that the first lens group includes two or more lens components having at least positive and negative lens components. Furthermore, it is preferable from the viewpoint of suppressing spherical aberration that the lens component constituting the first lens group includes an aspherical lens.

(2) Second Lens Group

The second lens group is a lens group disposed closer to the photoacoustic element than the first lens group, and has positive refractive power. The second lens group may be composed of at least one lens component, and may have positive refractive power as a whole. It is preferable from the viewpoint of increasing a diameter of the ray beam incident on the second lens group and increasing a numerical aperture (NA) of the ray directed to an object that the second lens group includes a lens component having negative refractive power. In addition, it is preferable from the viewpoint of suppressing spherical aberration that the lens component constituting the second lens group includes an aspherical lens.

(3) Photoacoustic Element

The photoacoustic element is disposed closest to the object in the condensing objective optical system, reflects an optical wave incident from the second lens group side toward the object, and transmits a photoacoustic wave emitted from the object that has absorbed the optical wave. The photoacoustic element is only required to be able to realize the above-described reflection and transmission, and, for example, can be configured by a metal layer, a first member bonded to one main face of the metal layer, and a second member bonded to the other main face of the metal layer.

In the photoacoustic element, the metal layer mainly reflects an optical wave and mainly transmits a photoacoustic wave. Such a metal layer can suppress a decrease in resolution due to occurrence of an axisymmetric aberration when an optical wave is transmitted through the metal layer. In addition, the metal layer can suppress a decrease in the SN ratio of the photoacoustic wave due to generation of mode conversion of the photoacoustic wave when the photoacoustic wave is reflected by the metal layer.

The configuration of the metal layer is not limited. For example, the metal layer may include only the main layer, or may further include a foundation layer formed on one or both main faces of the main layer in addition to the main layer. The foundation layer may be, for example, a metal layer in which the thickness of the thickest portion is 20 nm or less. It is preferable to have a foundation layer from the viewpoint of enhancing the bonding strength between each of the first member and the second member and the metal layer.

Examples of the metal material of the metal layer include aluminum (Al), titanium (Ti), silver (Ag), copper (Cu), gold (Au), platinum (Pt), and alloys containing these metals. These metal materials are preferable from the viewpoint of making the difference between the acoustic impedance of the first member and the second member and the acoustic impedance of the metal layer small.

The first member and the second member can be made of a glass material. The glass material can be appropriately determined within a range in which preferable characteristics as a photoacoustic element are exhibited. For example, it is preferable from the viewpoint of reducing the difference between the acoustic impedance of the first member and the second member and the acoustic impedance of the metal layer and reducing the reflectivity to the photoacoustic wave (increasing the transmittance) that the density of the glass material is 2.6×10³ kg/m³ or more. It is also preferable that the Poisson's ratio of the glass material is 2.1 or more from the above viewpoint. It is preferable from the viewpoint of detecting a photoacoustic wave having sufficient intensity in the photoacoustic device that the glass material has the above characteristics.

In addition, it is preferable from the viewpoint of realizing both the preferable density and Poisson's ratio that one or both of the first member and the second member are made of optical glass or ceramics. Examples of ceramics include sapphire and alumina. When only one of the first member and the second member is made of optical glass or ceramics, it is preferable that a member on which an optical wave is incident among the first member and the second member is made of optical glass or ceramics.

It is preferable from the viewpoint of easily collecting the photoacoustic wave generated from the object by radiation of the optical wave that the photoacoustic element has a concave face portion facing the object. When the photoacoustic element has a convex face portion facing the object side, the photoacoustic wave reaching the photoacoustic element tends to diffuse.

In addition, it is preferable from the viewpoint of condensing the optical wave reflected by the metal layer toward the object that the photoacoustic element has a concave face portion facing the object side. By condensing the optical wave at the metal layer, the beam waist diameter of the optical wave irradiating the object can be reduced, and as a result, the resolution of the photoacoustic device can be further improved. In addition, by condensing the optical wave at the metal layer, the distance from the photoacoustic element to the object can be further reduced, and miniaturization of the condensing objective optical system and the photoacoustic device due to the reduction of the distance can be realized.

The concave face portion can be formed on a face, of the photoacoustic element, facing the object side. For example, the concave face portion may be formed by a main face, of the metal layer, facing the object, or may be formed by a face, facing the object, of a member, among the first member and the second member, disposed between the metal layer and the object. The concave face portion is preferably a paraboloid concave face portion from the viewpoint of collecting optical waves.

1-2. Operation During Focusing

In the present embodiment, the second lens group performs focusing. It is preferable to perform focusing by moving the second lens group along the optical axis while the first lens is fixed with respect to the light source from the viewpoint of achieving high-speed focusing. It is preferable from the viewpoint of focusing on a target object at a shallow depth position that the second lens group moves toward the first lens group (toward light source) to perform focusing. It is preferable from the viewpoint of focusing on a target object at a deep position that the second lens group moves toward the photoacoustic element (toward object) to perform focusing.

1-3. Expressions Expressing Conditions of Optical System

The condensing objective optical system according to the embodiment of the present invention preferably adopts the above-described configuration and satisfies at least one or a plurality of the following expressions.

The condensing objective optical system in the embodiment of the present invention preferably includes at least one lens satisfying the following Expressions (1) and (2):

$$\begin{matrix}vd > 64 & ( 1 ) \\ 0.294 \leq f \text{/} fvMAX < 2.140 & ( 2 )\end{matrix}$$

where

νd is an Abbe number with respect to d Line,

f is a focal distance of the condensing objective optical system when the object distance is longest, and

fνMAX is focal distance of the lens whose vd is maximum.

Expression (1) defines a material for correction of chromatic aberration in the condensing objective optical system. It is preferable from the viewpoint of suppressing the occurrence of chromatic aberration that the condensing objective optical system has a lens satisfying Expression (1). When the Abbe number of the lens falls below the range of Expression (1), it may be difficult to sufficiently suppress the occurrence of chromatic aberration. The Abbe number of the lens can be appropriately determined from the range represented by Expression (1) from the viewpoint of sufficiently suppressing the occurrence of chromatic aberration.

Expression (2) defines the focal distance for correction of chromatic aberration in the condensing objective optical system. The condensing objective optical system preferably includes a lens satisfying Expression (2) from the viewpoint of suppressing the occurrence of chromatic aberration. When the ratio f/fνMAX of the focal distance of the condensing objective optical system to the focal distance of the lens whose νd is maximum is smaller than the range of the Expression (2) or larger than the range, in a case where the optical wave includes optical waves of two wavelengths, for example, two optical waves of an infrared ray having a wavelength of 1064 nm and an infrared ray having a wavelength of 700 nm, a deviation between the focal positions of these optical waves in the optical axis direction is large, and the resolution in the photoacoustic device may be insufficient. f/fνMAX is preferably 0.935 or more, or 1.905 or less from the viewpoint of suppressing the occurrence of the deviation between the focal positions in the case of including the optical waves of two wavelengths.

The condensing objective optical system in the embodiment of the present invention preferably satisfies the following Expression (3):

$$1.000 < Δ Z \text{/} Lf \leq 2.386 \qquad ( 3 )$$

where

Z is an amount of change in an object distance and

Δf is a movement distance of the second lens group.

Expression (3) defines an amount of change in the object distance and a focus movement amount. The “object distance” is a distance on the optical axis from a surface, of the photoacoustic element, on the object side to an imaging position at the object side in the condensing objective optical system. It is preferable from the viewpoint of suppressing the occurrence of spherical aberration of the condensing objective optical system and improving the light condensing performance in the condensing objective optical system that the condensing objective optical system satisfies Expression (3). When the ratio ΔZ/Lf of an amount of change in the object distance to the movement distance of the second lens group is smaller than the range of Expression (3), the amount of movement of the second lens group in focusing is small, so that focusing is lost. As a result, spherical aberration is large, and the light condensing performance may be insufficient. When ΔZ/Lf is larger than the range of Expression (3), the amount of movement of the second lens group in focusing is large, spherical aberration increases, and as a result, light condensing performance may be insufficient. From the viewpoint of enhancing the light condensing performance, ΔZ/Lf is more preferably 1.040 or more, still more preferably 1.308 or more. From the same viewpoint, ΔZ/Lf is more preferably 1.515 or less, still more preferably 1.333 or less.

The condensing objective optical system in the embodiment of the present invention preferably satisfies the following Expression (4) when a direction in which a pencil of light of an optical wave diverges is positive and a direction in which the pencil of light is condensed is negative:

$$- 4.0 ° \leq a 12 \leq 4.0 ° \qquad ( 4 )$$

where

a12 is a divergence angle of the pencil of light of the optical wave between first lens group and second lens group.

Expression (4) defines the divergence angle of the pencil of light incident on the second lens group. It is preferable from the viewpoint of appropriately converging the pencil of light of the optical wave when the second lens group is moved so that the object distance changes that the condensing objective optical system satisfies Expression (4). The performance for converging the pencil of light is also referred to as a “spot performance”. When the divergence angle a12 is smaller than the range of Expression (4), the spot performance may be insufficient particularly when the second lens group is moved in a direction in which the object distance is increased. When the divergence angle a12 is larger than the range of Expression (4), the spot performance may be insufficient when the second lens group is moved so as to change the object distance. From the viewpoint of sufficiently exhibiting spot performance, the divergence angle a12 is more preferably −2.0 or more, still more preferably −0.2 or more. From the same viewpoint, the divergence angle a12 is more preferably 2.0 or less, and still more preferably 0.2 or less.

In the condensing objective optical system according to the embodiment of the present invention, the medium reaching the target object is preferably based on the medium satisfying the following Expression (5):

$$nWD < 1.53 \qquad ( 5 )$$

where

nWD is a refractive index of a medium with respect to d Line.

Expression (5) defines the refractive index of the medium for the object that is to be irradiated with the optical wave and the photoacoustic wave from which is to be detected in the condensing objective optical system. Expression (5) more specifically defines a substance from air to optical glass. As described above, the “medium” is a portion, of the (target) object, through which the optical wave from the photoacoustic element passes. It is preferable from the viewpoint of enhancing the versatility of the condensing objective optical system that the condensing objective optical system is configured based on the medium satisfying Expression (5), and the imaging technique in the photoacoustic device can be applied to a wide range of fields from laser processing, which is a conventional mainstream, to observation of the inside of a living body.

The condensing objective optical system applicable to such a medium can be configured by appropriately adjusting the optical characteristics within the range defined by the expressions in the present embodiment. For example, it is possible to configure a condensing objective optical system applicable to a medium having a large value of Expression (5) by increasing the value of Expression (3) described above.

The condensing objective optical system in the embodiment of the present invention preferably satisfies the following Expression (6):

$$1.729 \leq np \leq 2.051 \qquad ( 6 )$$

where

np is a refractive index of a portion, of the photoacoustic element, through which an optical wave passes with respect to d Line.

Expression (6) defines the refractive index of a portion, of the photoacoustic element, through which an optical wave passes, for example, the glass material of the first member or the second member described above. It is preferable from the viewpoint of enhancing the transparency of the photoacoustic wave that the condensing objective optical system satisfies Expression (6). When the refractive index np of the portion of the photoacoustic element composed of the glass material is smaller than the range of Expression (6) or larger than the range, transmission of a photoacoustic wave generated by irradiation of an optical wave through the photoacoustic element is insufficient, and detection sensitivity of the photoacoustic wave in the photoacoustic device may be insufficient. From the viewpoint of enhancing the transmittance of the photoacoustic wave in the photoacoustic element, the refractive index np may be appropriately determined from the range represented by Expression (6).

2. Photoacoustic Device

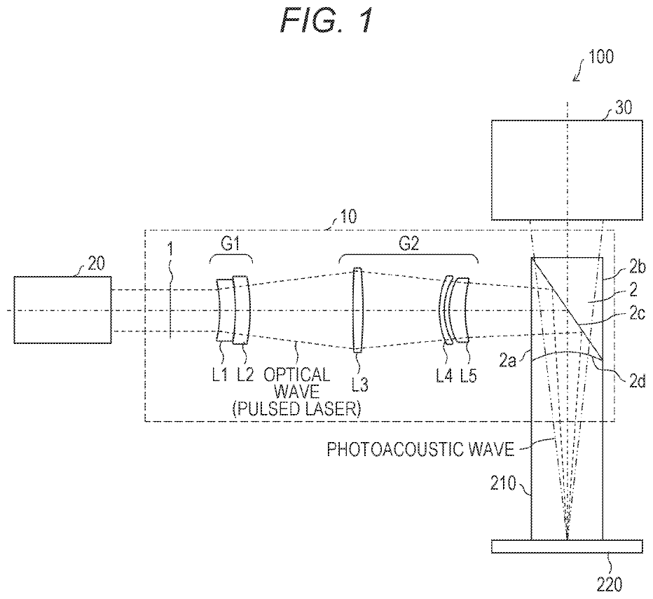

A photoacoustic device according to an embodiment of the present invention includes the above-described condensing objective optical system, and detects a photoacoustic wave emitted by the object that has absorbed an optical wave emitted from the condensing objective optical system. FIG. 1 is a block diagram schematically illustrating a configuration of a photoacoustic device according to an embodiment of the present invention. As illustrated in FIG. 1, a photoacoustic device 100 includes a condensing objective optical system 10, a light source 20, and an acoustic wave detector 30.

The condensing objective optical system 10 includes a stop 1, a first lens group G1, a second lens group G2, and a photoacoustic element 2 in order from the light source side.

The stop 1 adjusts a radiation cross section of the optical wave output from light source 20 according to a size of a desired imaging region of a subject 220 that is a target object.

The first lens group G1 is disposed closer to the photoacoustic element 2 than the stop 1. The first lens group G1 includes two lenses L1 and L2, and the lenses L1 and L2 are fixed in the direction along the optical axis. First lens group G1 has negative refractive power as a whole.

The second lens group G2 is disposed between the first lens group G1 and the photoacoustic element 2. The second lens group G2 includes three lenses L3, L4, and L5, and each of the lenses L3, L4, and L5 is configured to be movable in the direction along the optical axis without changing a relative position in the direction along the optical axis. Second lens group G2 has positive refractive power as a whole. Second lens group G2 is a lens group movable along the optical axis, and is a focus group that performs focusing.

The photoacoustic element 2 includes a first member 2a, a second member 2b, and a metal layer 2c. Each of the first member 2a and the second member 2b has a shape obtained by cutting a glass material having a regular quadrangular prism along a height direction at a diagonal line of a top face. One of the square side faces of the first member 2a faces the light source 20, and the other side face faces the subject 220.

The remaining side face of the first member 2a is joined to that of the second member 2b via the metal layer 2c. One of the square side faces of the second member 2b faces the acoustic wave detector 30.

The metal layer 2c has, for example, a three-layer structure including a silver main layer and a foundation layer that is a titanium layer formed on each of both main faces of the silver main layer.

Note that the photoacoustic element 2 has a concave face portion 2d at a face facing the subject 220. The concave face portion 2d is a portion where the other side face of the first member 2a is formed in a concave face shape recessed facing the subject 220.

The light source 20 is configured to generate an optical wave, and is, for example, a pulsed laser. The wavelength of the pulsed laser may be set according to the spectral absorption characteristics of the subject 220. The pulse width of the pulsed laser generated by the light source 20 may be appropriately set in a range of several picoseconds to several hundred nanoseconds. As an example, a pulsed laser having a wavelength of 1064 nm and a pulse width of 10 ns can be used.

The light source 20 may be a light source that outputs two or more kinds of optical waves having different wavelengths. In this case, the optical wave output from the light source 20 can be an optical wave adapted to the spectral absorption characteristics of each of the two or more kinds of subjects 220. Note that the light source 20 may be a light source of pulsed laser whose wavelength of the oscillating pulsed laser is variable. In this case, the difference in absorption characteristics of each part of the subject 220 can be reflected in the image obtained by detecting the photoacoustic wave.

The acoustic wave detector 30 is configured to convert a photoacoustic wave into an electrical signal. The acoustic wave detector 30 is disposed to face the subject 220 via the photoacoustic element 2, and has an axis of a photoacoustic wave along a direction orthogonal to the optical axis of the light source 20 and the condensing objective optical system 10. The acoustic wave detector 30 can be appropriately selected from a device according to the subject 220 and the acoustic wave characteristics. As the acoustic wave detector 30, for example, an acoustic transducer can be used.

Next, detection of a photoacoustic wave in the present embodiment will be described using a case where a tissue in a living body is the subject 220 as an example. The photoacoustic device 100 is disposed such that the concave face portion 2d of the photoacoustic element 2 contacts the surface of the living body.

The optical wave output from the light source 20 is adjusted by the stop 1 so as to have a diameter of the pencil of light suitable for imaging the subject 220. The diameter of the pencil of light of the optical wave having passed through the stop 1 is enlarged by passing through the first lens group G1, and is appropriately reduced according to the positive refractive power of the second lens group G2 and the position of the second lens group on the optical axis by passing through the second lens group G2. With such power arrangement of the lens group, the chromatic aberration of the optical wave is corrected, and in a case where the light source 20 outputs two types of optical waves having different wavelengths, achromatization of optical waves having two or more wavelengths is appropriately performed.

Next, the optical wave enters the first member 2a in the photoacoustic element 2, is reflected by the surface of the metal layer 2c, and is output toward the subject 220. The optical wave passes through a medium 210 of the living body while the diameter of the pencil of light thereof is gradually reduced, and is focused on the subject 220.

Subject 220 that has absorbed the optical wave generates a photoacoustic wave. The photoacoustic wave passes through the medium 210, passes through the first member 2a, the metal layer 2c, and the second member 2b of the photoacoustic element 2 in this order from the concave face portion 2d, and enters the acoustic wave detector 30. The acoustic wave detector 30 detects the incident photoacoustic wave. An image is formed according to the signal of the detection result of the acoustic wave detector 30, whereby the subject 220 is imaged.

Since the photoacoustic device 100 includes the light source 20, it is possible to output an optical wave suitable for imaging the subject 220. In addition, since the stop 1 is provided, the optical wave has the radiation cross section that can be appropriately adjusted according to the subject 220 before passing through the first lens group G1 and the second lens group G2. Furthermore, since the second lens group G2 is provided, an optical wave having an appropriate diameter of the pencil of light according to the focal distance to the subject 220 and the medium 210 can be output to the photoacoustic element.

Since the photoacoustic device 100 has the metal layer 2c, it is possible to reflect the optical wave toward the subject 220. In addition, since the photoacoustic element 2 has the concave face portion 2d, the optical wave is easily condensed toward the subject 220, and the photoacoustic wave is sufficiently easily generated in the subject 220.

In the photoacoustic device 100, since the concave face portion 2d suppresses the divergence of the photoacoustic wave generated in the subject 220 on the surface of the photoacoustic element 2, the reception of the photoacoustic wave is promoted. In addition, since the photoacoustic wave passes through the first member 2a, the metal layer 2c, and the second member 2b described above, attenuation of the photoacoustic wave generated in the subject 220 is suppressed, and the acoustic wave detector 30 can detect the photoacoustic wave having sufficient intensity. Therefore, it is possible to form an image with higher resolution in the subsequent imaging.

The photoacoustic device 100 includes the condensing objective optical system 10 including the first lens group G1 and the second lens group G2 having the above-described characteristics. Therefore, it is possible to realize a photoacoustic optical system that can also be used to observe the inside of the living body, and it is possible to detect a photoacoustic wave that can also be used to observe the inside of the living body.

Note that the photoacoustic device according to the embodiment of the present invention may further have other configurations than those described above within a range in which the effects of the present embodiment can be obtained. For example, the photoacoustic device in the embodiment of the present invention may further include a function generator (not illustrated) that controls the light source. Furthermore, the photoacoustic device may further include a fast Fourier transform (FFT) analyzer (not illustrated) that processes the electrical signal obtained by the acoustic wave detector.

Furthermore, the photoacoustic device may further include a stage for scanning a focal point of the optical wave on the subject. A water tank that accommodates a subject can be placed on the stage, and the subject can be moved together with the water tank. In this case, the photoacoustic wave is emitted to a subject in the water, and the photoacoustic wave is emitted from the subject in the water. Even in such an embodiment, the photoacoustic device according to the embodiment of the present invention can detect the photoacoustic wave with a sufficiently high resolution so that an image with a high resolution can be formed.

Furthermore, the photoacoustic device may further include an acoustic matching material (not illustrated) interposed between the subject and the photoacoustic element. Furthermore, the photoacoustic device may further include a plurality of condensing objective optical systems and a switching mechanism (not illustrated) that switches the condensing objective optical systems used from the plurality of condensing objective optical systems. In this case, when two or more condensing objective optical systems are used in combination, it is also possible to superimpose images obtained through individual condensing objective optical systems.

In addition, the function generator, the FFT analyzer, the stage, and the switching mechanism described above may be connected to a computer (not illustrated). In this case, the computer can be used to control the function generator, the stage, and the switching mechanism described above, and to generate an image of the inside of the subject from the output of the FFT analyzer.

The present invention is not limited to the above-described embodiments, and various modifications can be made within the scope indicated in the claims. Embodiments obtained by appropriately combining technical means disclosed in different embodiments are also included in the technical scope of the present invention.

### EXAMPLES

Examples of the condensing objective optical system of the present invention will be described with reference to the drawings. In the drawings illustrating the optical configuration in each example, “STO” represents a stop, “L1”, “L2”, “L3”, “L4”, and “L5” represent lenses, “P” represents a prism which is a photoacoustic element, “G1” represents a first lens group, and “G2” represents a second lens group. In each Example, the second lens group G2 is a focus group that moves along the optical axis. In addition, “WD” in the drawing represents a portion of the object distance, and is a portion of the medium with which a space between the target object that is the subject by photoacoustic imaging and the prism of the condensing objective optical system is substantially filled. “WD” is, for example, a tissue of a living body or an air layer. Note that the intersection point between the optical axis and the edge, of WD, away from the prism P is an imaging position (object point) on the object side in the optical system of the Examples, and usually represents the position of the target object.

Further, in the drawing illustrating aberrations in the respective Examples, the left side graph on the sheet of the figure indicates spherical aberration Y (μm) in the direction perpendicular to the pupil, and the right side graph thereof indicates spherical aberration X (μm) in the direction horizontal to the pupil. A solid line indicates the infrared ray having a reference wavelength of 1064 nm of the master lens, and a broken line indicates the infrared ray having a wavelength of 700 nm. In addition, the graph below indicates longitudinal chromatic aberration. The vertical axis represents a wavelength, and the horizontal axis represents a focus position. A solid line represents characteristics of the infrared ray at a focus position from a wavelength of 700 nm to a reference wavelength of 1064 nm of the master lens. Here, the master lens in each Example is a condensing objective optical system.

Furthermore, numerical data regarding the optical characteristics of each Example is shown in the table. In the table of each Example, “R” represents the radius of curvature (mm) of the spherical lens (SPH), and “D” represents the thickness of the lens on the optical axis or the interval (mm) between the lenses. “N” represents the refractive index of the lens with respect to d Line, and “ν” represents the Abbe number of the lens with respect to d Line. As in the drawing, “STO” represents the stop, and “P” represents the prism.

In addition, “(1)” in the table is a distance between facing surfaces of lenses facing each other among the lenses of the first lens group and the lenses of the second lens group. “(2)” in the table is a distance between facing surfaces of the lens facing the prism among the lenses of the second lens group and the prism. “(3)” in the table represents the object distance, and is the distance on the optical axis from the surface, of the prism, closest to the object side to the object point.

### Example 1

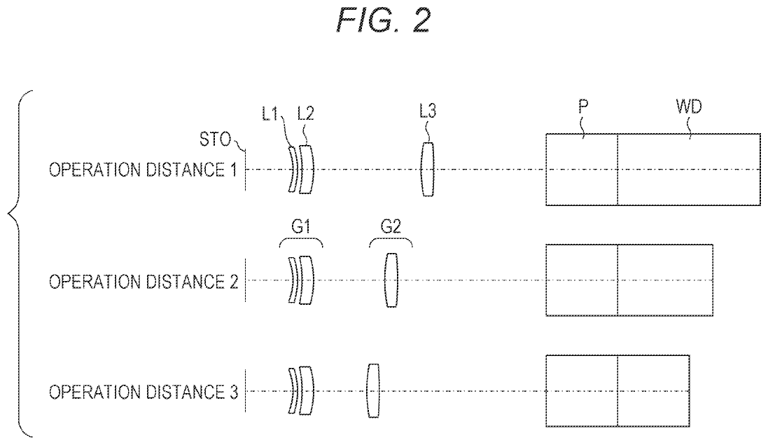

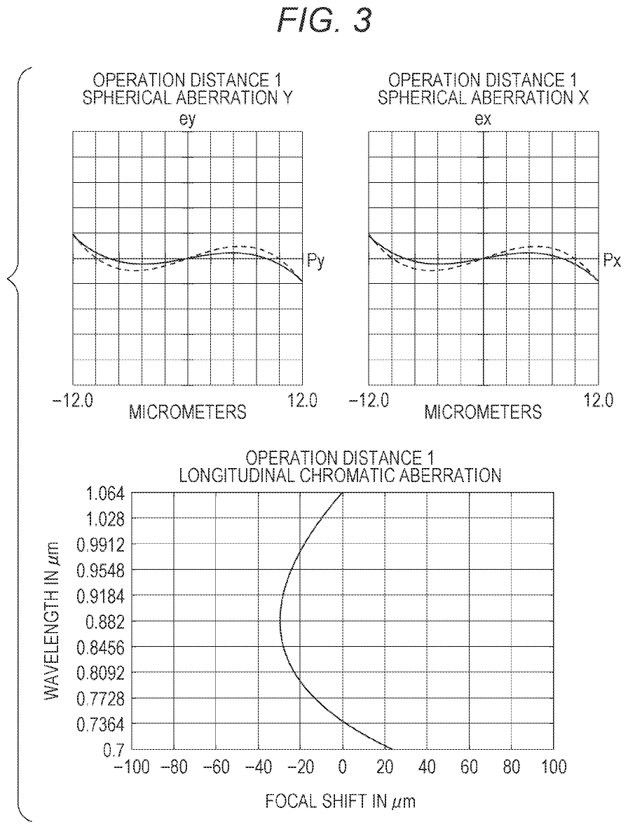

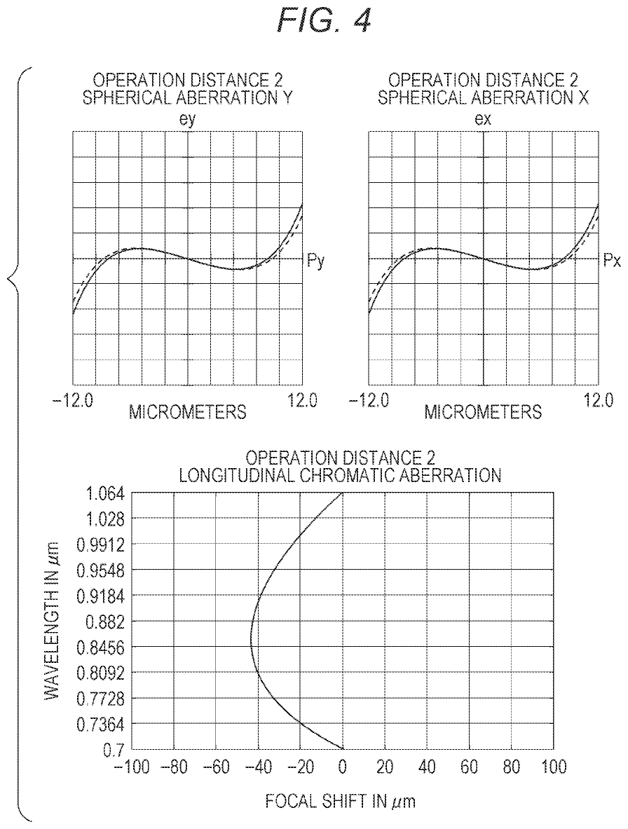

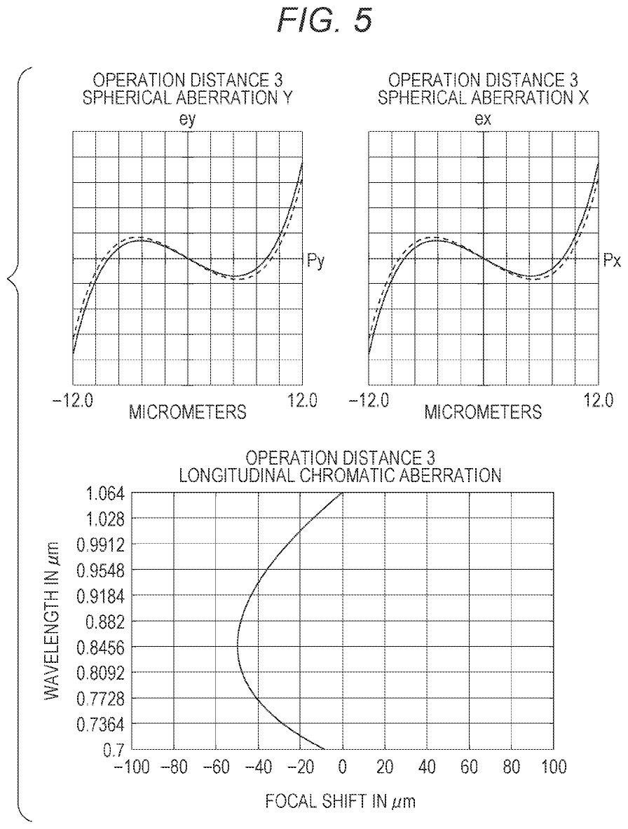

FIG. 2 is a diagram schematically illustrating an optical configuration according to the object distance of the condensing objective optical system of Example 1 of the present invention. FIG. 3 is a diagram illustrating a graph of spherical aberration and a graph of longitudinal chromatic aberration at the operation distance 1 in Example 1 of the present invention. FIG. 4 is a diagram illustrating a graph of spherical aberration and a graph of longitudinal chromatic aberration at the operation distance 2 in Example 1 of the present invention. FIG. 5 is a diagram illustrating a graph of spherical aberration and a graph of longitudinal chromatic aberration at the operation distance 3 in Example 1 of the present invention. Table 1 shows the optical data in Example 1, and Table 2 shows the object distance in Example 1, and the focal distance, the F number (FNo), the distance between the lens groups, and the distance between the lens and the prism of the condensing objective optical system corresponding thereto.

The “operation distance” is a distance on the optical axis from a position of the second lens group of the condensing objective optical system when the object distance is longest to a position of the second lens group when the object distance is specific (for example, (3) in the following Table 2). Furthermore, as illustrated in FIG. 2, the prism P in the present example faces the portion WD side of the object distance in a plane.

TABLE 11

| Face | | | | | |
|---|---|---|---|---|---|
| number | | R | D | N | ν |
| 1 | STO | 0.000 | 10.000 | | |
| 2 | | −11.612 | 1.000 | 2.003 | 19.317 |
| 3 | | −13.968 | 1.000 | | |
| 4 | | −16.744 | 2.401 | 1.487 | 70.441 |
| 5 | | −14.365 | (1) | | |
| 6 | | 29.629 | 2.665 | 1.437 | 95.100 |
| 7 | | −67.602 | (2) | | |
| 8 | P | 0.000 | 15.000 | 1.911 | 35.250 |
| 9 | | 0.000 | (3) | 1.333 | 55.794 |

TABLE 2

| | Operation | Operation | Operation |
|---|---|---|---|
| | distance 1 | distance 2 | distance 3 |
| Focal distance | 46.957 | 48.352 | 49.083 |
| FNo | 5.217 | 5.372 | 5.454 |
| (1) | 22.599 | 14.749 | 10.818 |
| (2) | 24.737 | 32.587 | 36.518 |
| (3) | 30.000 | 20.000 | 15.000 |

### Example 2

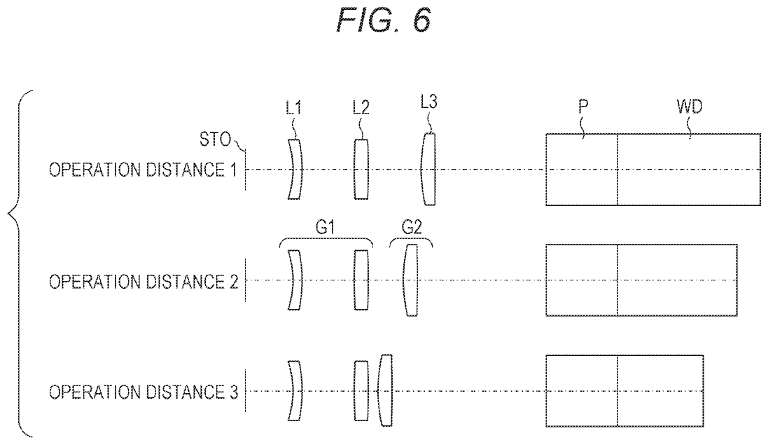

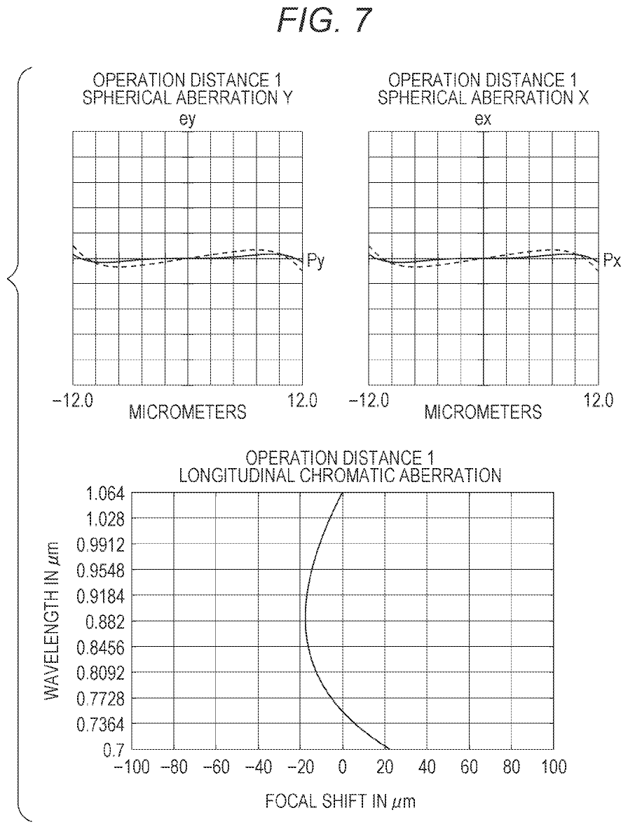

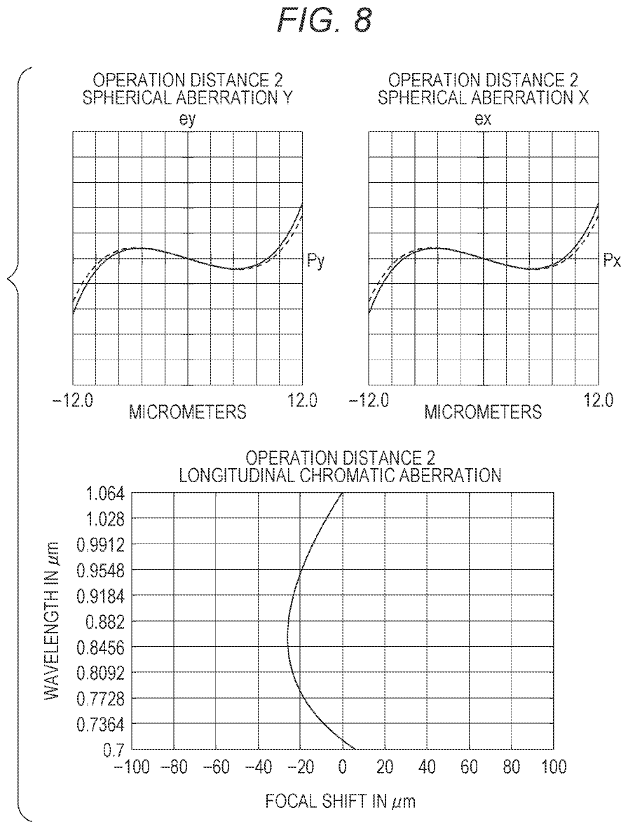

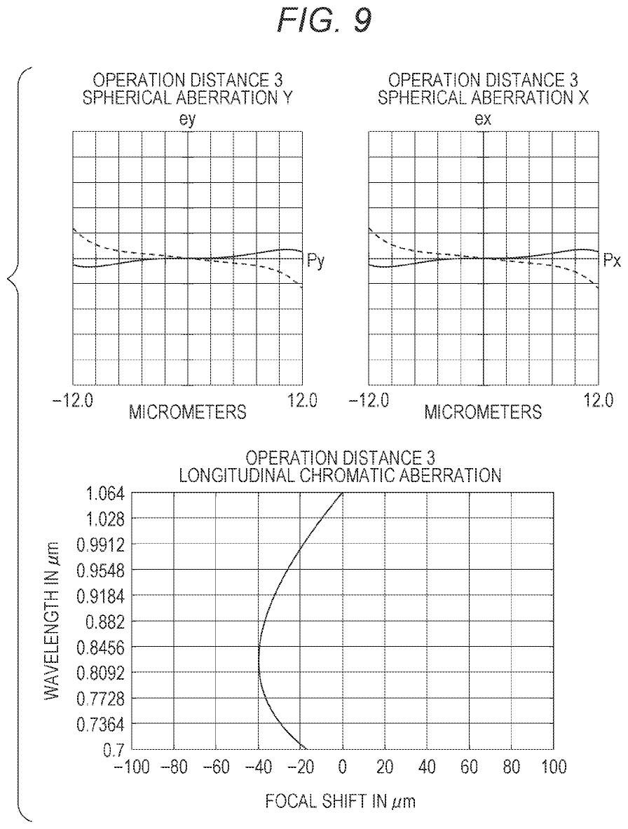

FIG. 6 schematically illustrates an optical configuration of the condensing objective optical system of Example 2, and FIGS. 7 to 9 illustrate graphs of spherical aberration and graphs of longitudinal chromatic aberration at the operation distances 1 to 3 in Example 2. Table 3 shows the optical data in Example 2, and Table 4 shows the object distance in Example 2, and the focal distance, the F number (FNo), the distance between the lens groups, and the distance between the lens and the prism of the condensing objective optical system corresponding thereto.

TABLE 3

| Face | | | | | |
|---|---|---|---|---|---|
| number | | R | D | N | ν |
| 1 | STO | 0.000 | 10.000 | | |
| 2 | | −19.799 | 2.000 | 1.923 | 20.880 |
| 3 | | −28.200 | 10.896 | | |
| 4 | | 104.948 | 3.000 | 1.500 | 81.608 |
| 5 | | −120.776 | (1) | | |
| 6 | | 31.612 | 3.000 | 1.550 | 75.496 |
| 7 | | −289.948 | (2) | | |
| 8 | P | 0.000 | 15.000 | 1.911 | 35.250 |
| 9 | | 0.000 | (3) | 1.333 | 55.794 |

TABLE 4

| | Operation | Operation | Operation |
|---|---|---|---|
| | distance 1 | distance 2 | distance 3 |
| Focal distance | 46.832 | 47.064 | 47.392 |
| FNo | 5.203 | 5.229 | 5.266 |
| (1) | 11.103 | 7.310 | 1.999 |
| (2) | 24.036 | 27.830 | 33.140 |
| (3) | 30.000 | 25.000 | 18.000 |

### Example 3

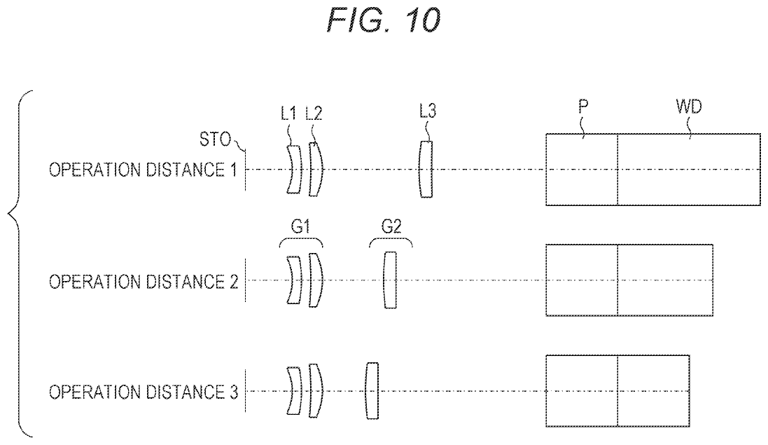

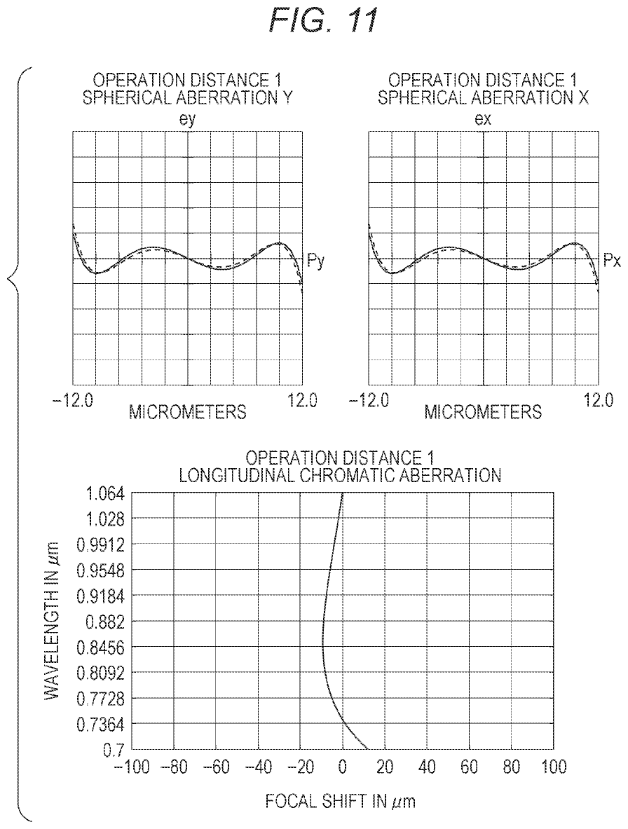

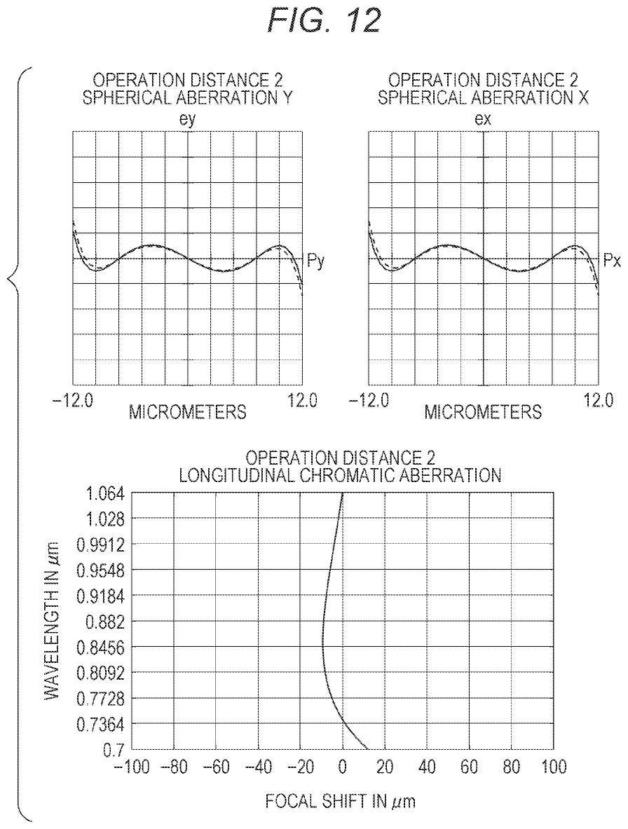

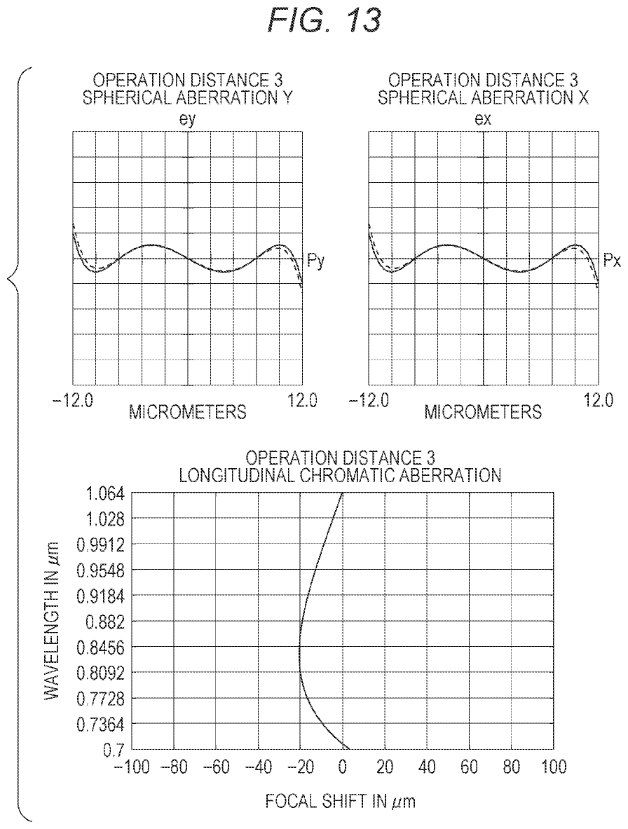

FIG. 10 schematically illustrates an optical configuration of the condensing objective optical system of Example 3, and FIGS. 11 to 13 illustrate graphs of spherical aberration and graphs of longitudinal chromatic aberration at the operation distances 1 to 3 in Example 3. Table 5 shows the optical data in Example 3, and Table 6 shows the object distance in Example 3, and the focal distance, the F number (FNo), the distance between the lens groups, and the distance between the lens and the prism of the condensing objective optical system corresponding thereto.

TABLE 5

| Face | | | | | |
|---|---|---|---|---|---|
| number | | R | D | N | ν |
| 1 | STO | 0.000 | 10.000 | | |
| 2 | | −10.259 | 1.936 | 1.752 | 25.048 |
| 3 | | −22.638 | 1.997 | | |
| 4 | | −45.984 | 2.401 | 1.500 | 81.608 |
| 5 | | −13.161 | (1) | | |
| 6 | | 41.145 | 2.665 | 1.900 | 37.372 |
| 7 | | 827.970 | (2) | | |
| 8 | P | 0.000 | 15.000 | 1.911 | 35.250 |
| 9 | | 0.000 | (3) | 1.333 | 55.794 |

TABLE 6

| | Operation | Operation | Operation |
|---|---|---|---|
| | distance 1 | distance 2 | distance 3 |
| Focal distance | 42.730 | 43.445 | 43.812 |
| FNo | 4.748 | 4.827 | 4.772 |
| (1) | 20.666 | 13.019 | 9.197 |
| (2) | 22.628 | 30.275 | 34.097 |
| (3) | 30.000 | 20.000 | 15.000 |

### Example 4

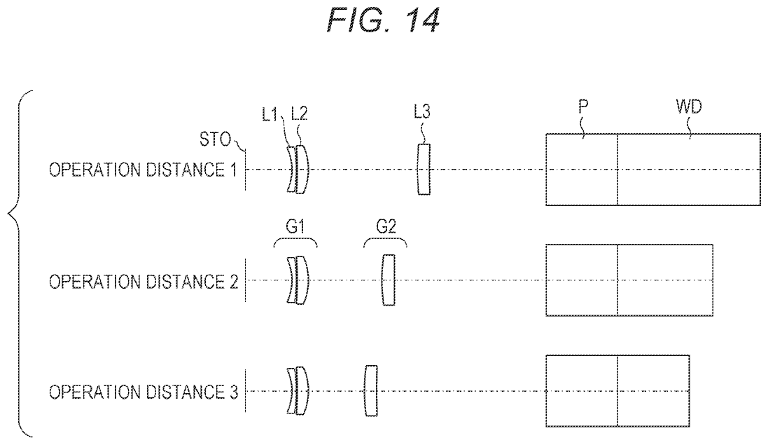

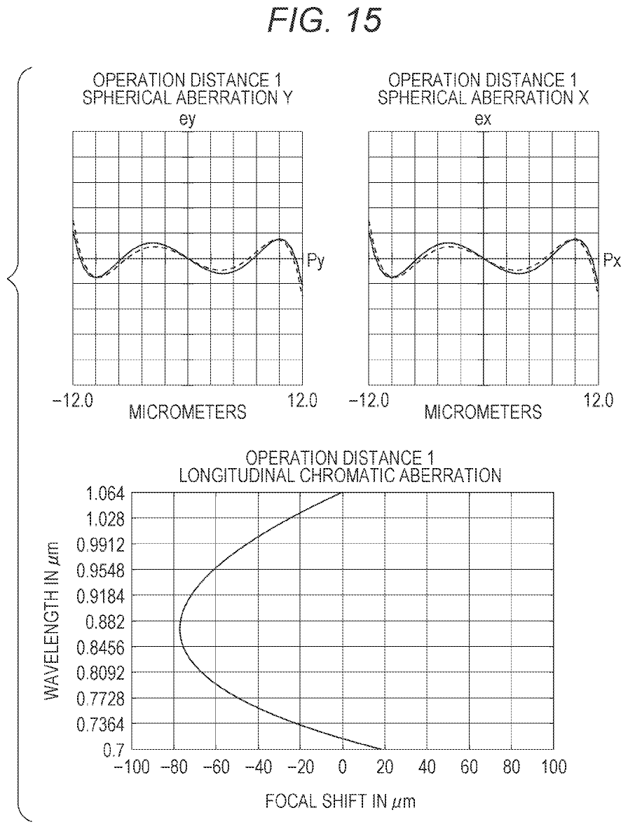

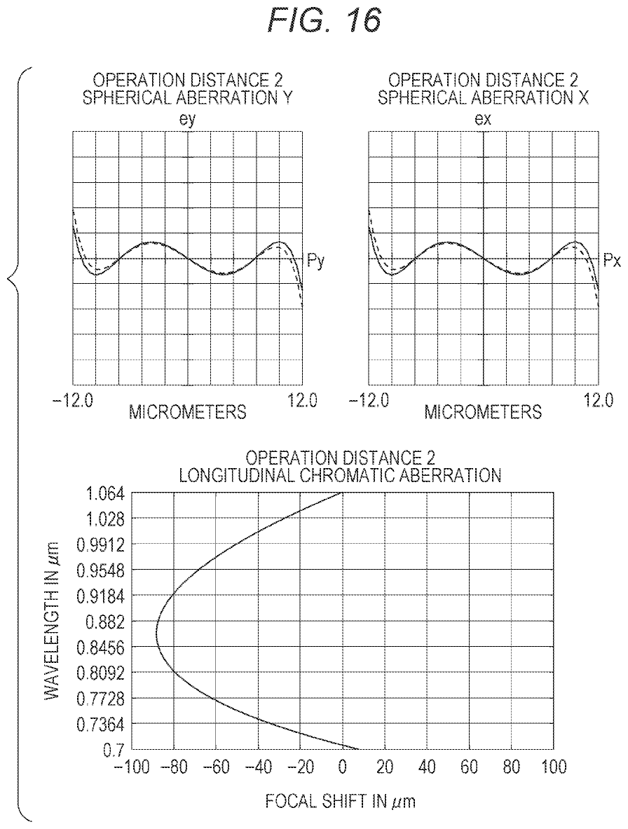

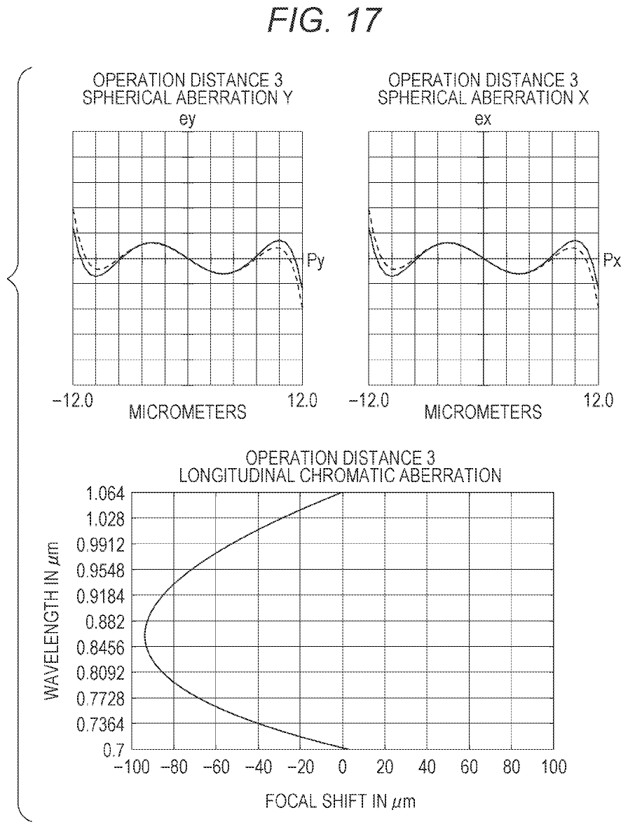

FIG. 14 schematically illustrates an optical configuration of the condensing objective optical system of Example 4, and FIGS. 15 to 17 illustrate graphs of spherical aberration and graphs of longitudinal chromatic aberration at the operation distances 1 to 3 in Example 4. Table 7 shows the optical data in Example 4, and Table 8 shows the object distance in Example 4, and the focal distance, the F number (FNo), the distance between the lens groups, and the distance between the lens and the prism of the condensing objective optical system corresponding thereto.

TABLE 7

| Face | | | | | |
|---|---|---|---|---|---|
| number | | R | D | N | ν |
| 1 | STO | 0.000 | 10.000 | | |
| 2 | | −10.513 | 1.000 | 1.805 | 25.456 |
| 3 | | −24.106 | 0.150 | | |
| 4 | | −96.428 | 2.401 | 1.4875 | 70.441 |
| 5 | | −12.291 | (1) | | |
| 6 | | 47.344 | 2.665 | 1.954 | 32.319 |
| 7 | | 7726.979 | (2) | | |
| 8 | P | 0.000 | 15.000 | 1.911 | 35.250 |
| 9 | | 0.000 | (3) | 1.333 | 55.794 |

TABLE 8

| | Operation | Operation | Operation |
|---|---|---|---|
| | distance 1 | distance 2 | distance 3 |
| Focal distance | 48.081 | 48.668 | 48.967 |
| FNo | 5.342 | 5.408 | 5.441 |
| (1) | 23.448 | 15.841 | 12.040 |
| (2) | 23.889 | 31.496 | 35.296 |
| (3) | 30.000 | 20.000 | 15.000 |

### Example 5

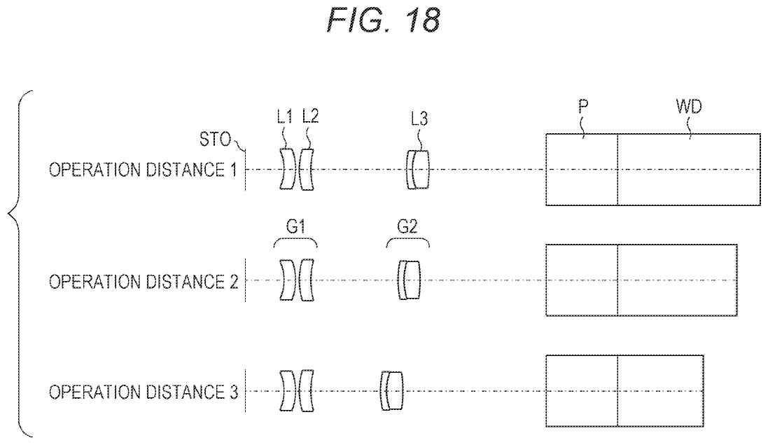

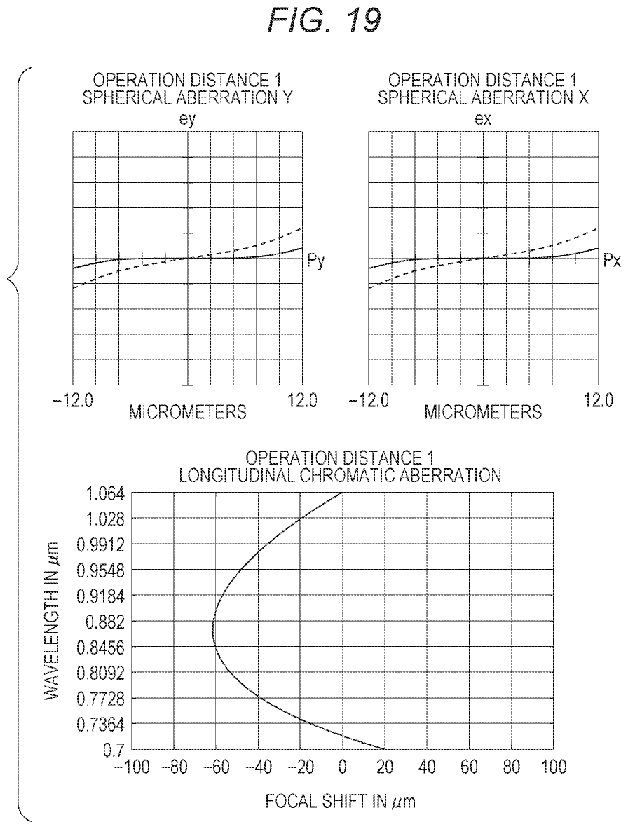

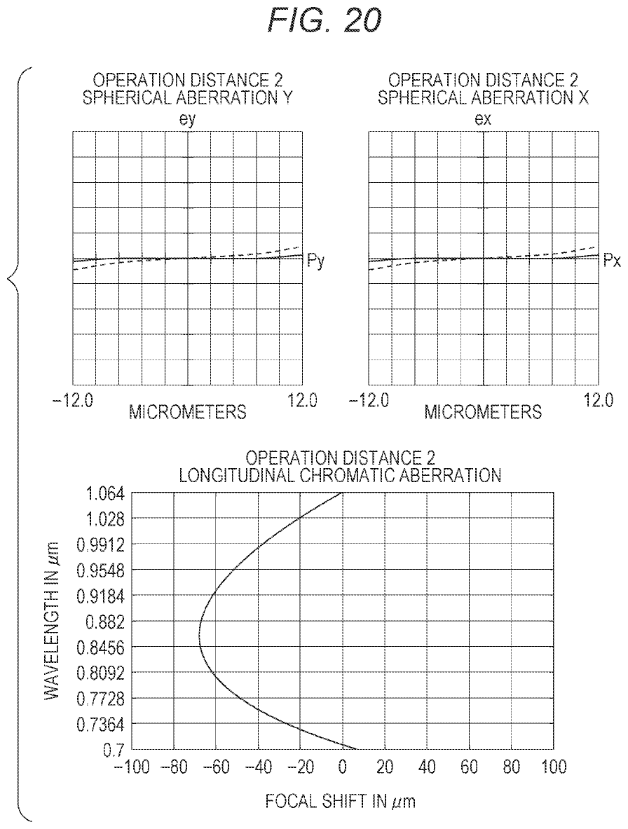

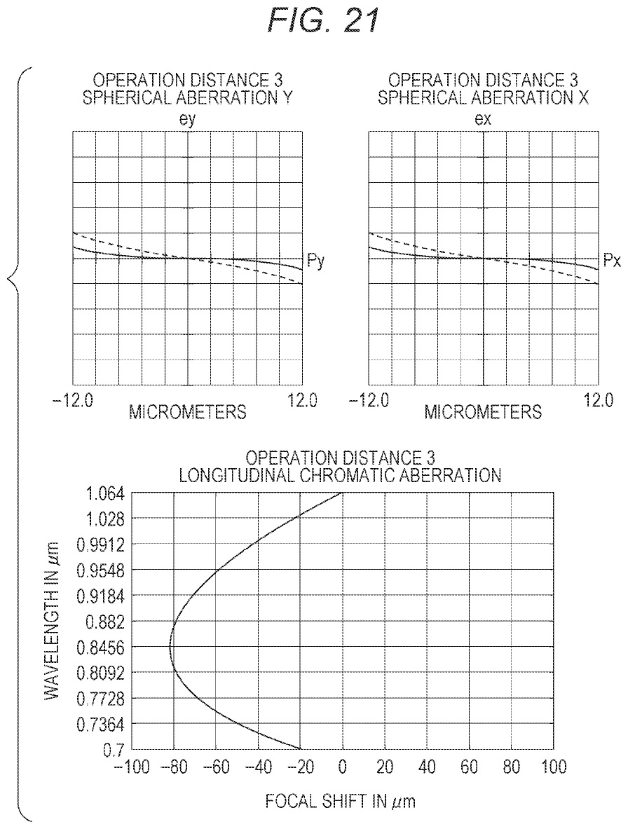

FIG. 18 schematically illustrates an optical configuration of the condensing objective optical system of Example 5, and FIGS. 19 to 21 illustrate graphs of spherical aberration and graphs of longitudinal chromatic aberration at the operation distances 1 to 3 in Example 5. Table 9 shows the optical data in Example 5, and Table 10 shows the object distance in Example 5, and the focal distance, the F number (FNo), the distance between the lens groups, and the distance between the lens and the prism of the condensing objective optical system corresponding thereto.

TABLE 9

| Face | | | | | |
|---|---|---|---|---|---|
| number | | R | D | N | ν |
| 1 | STO | 0.000 | 10.000 | | |
| 2 | | −11.105 | 2.949 | 2.001 | 29.135 |
| 3 | | −13.902 | 1.000 | | |
| 4 | | 17.465 | 3.000 | 1.673 | 32.171 |
| 5 | | 20.627 | (1) | | |
| 6 | | 26.505 | 1.500 | 1.847 | 23.623 |
| 7 | | 13.888 | 4.229 | 1.517 | 64.198 |
| 8 | | −35.901 | (2) | | |
| 9 | P | 0.000 | 15.000 | 1.911 | 35.250 |
| 10 | | 0.000 | (3) | 1.333 | 55.794 |

TABLE 10

| | Operation | Operation | Operation |
|---|---|---|---|
| | distance 1 | distance 2 | distance 3 |
| Focal distance | 41.019 | 41.016 | 41.015 |
| FNo | 4.558 | 4.557 | 4.557 |
| (1) | 24.770 | 22.513 | 18.000 |
| (2) | 13.928 | 16.185 | 20.698 |
| (3) | 23.000 | 20.000 | 14.000 |

### Example 6

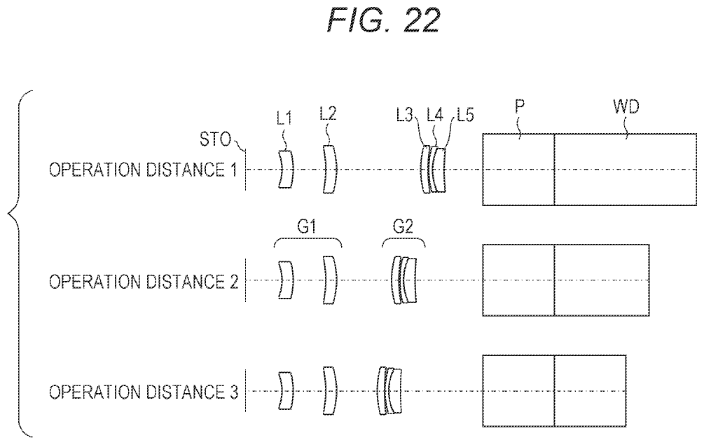

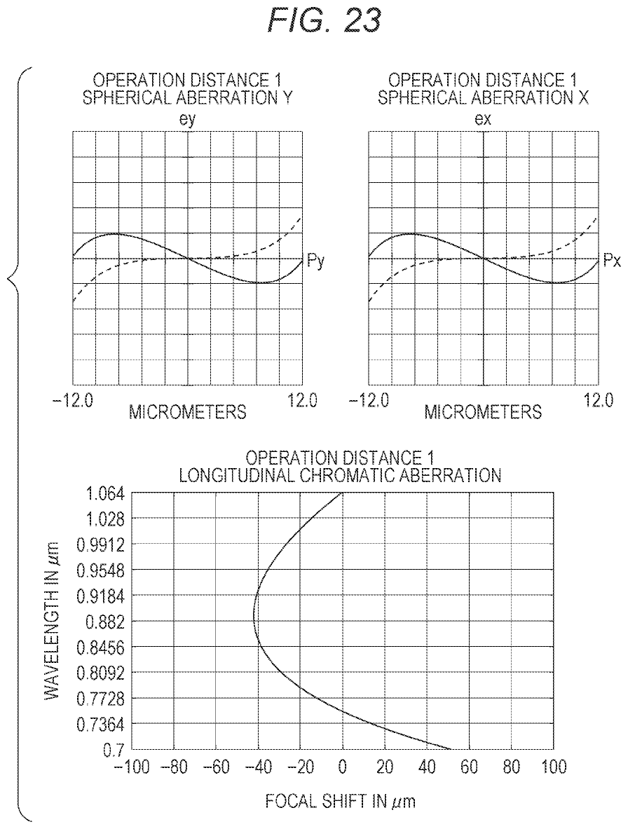

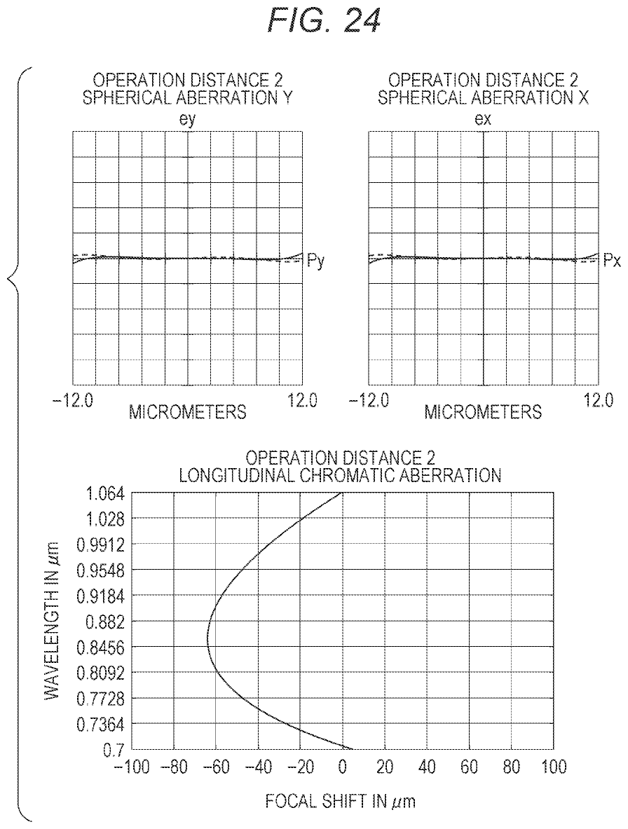

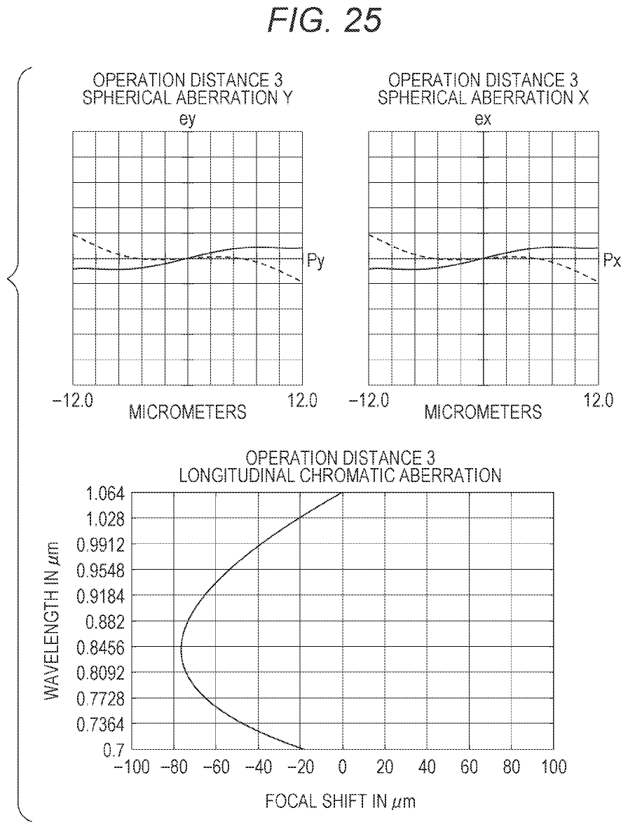

FIG. 22 schematically illustrates an optical configuration of the condensing objective optical system of Example 6, and FIGS. 23 to 25 illustrate graphs of spherical aberration and graphs of longitudinal chromatic aberration at the operation distances 1 to 3 in Example 6. Table 11 shows the optical data in Example 6, and Table 12 shows the object distance in Example 6, and the focal distance, the F number (FNo), the distance between the lens groups, and the distance between the lens and the prism of the condensing objective optical system corresponding thereto.

TABLE 11

| Face | | | | | |
|---|---|---|---|---|---|
| number | | R | D | N | ν |
| 1 | STO | 0.000 | 10.000 | | |
| 2 | | −12.579 | 3.000 | 1.946 | 17.984 |
| 3 | | −20.399 | 9.000 | | |
| 4 | | −28.639 | 3.000 | 1.954 | 32.319 |
| 5 | | −20.185 | (1) | | |
| 6 | | 24.559 | 2.000 | 1.773 | 49.624 |
| 7 | | 40.870 | 0.200 | | |
| 8 | | 23.370 | 1.000 | 1.805 | 25.456 |
| 9 | | 13.471 | 3.200 | 1.517 | 64.198 |
| 10 | | 89.072 | (2) | | |
| 11 | P | 0.000 | 15.000 | 1.911 | 35.250 |
| 12 | | 0.000 | (3) | 1.333 | 55.794 |

TABLE 12

| | Operation | Operation | Operation |
|---|---|---|---|
| | distance 1 | distance 2 | distance 3 |
| Focal distance | 33.369 | 33.369 | 33.369 |
| FNo | 3.708 | 3.708 | 3.708 |
| (1) | 22.960 | 14.998 | 11.018 |
| (2) | 10.586 | 18.548 | 22.528 |
| (3) | 30.594 | 20.000 | 14.703 |

### Example 7

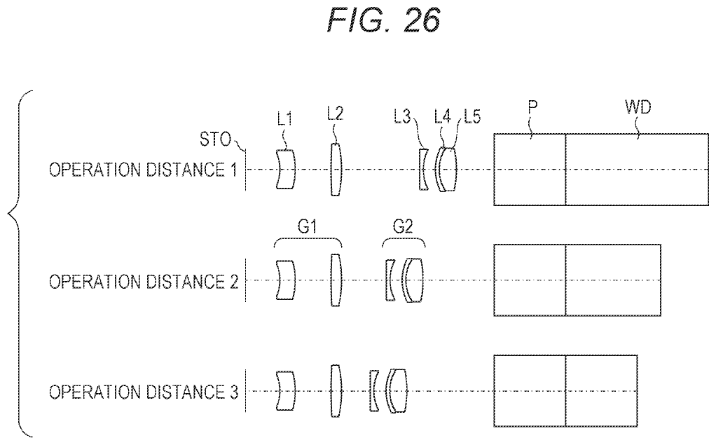

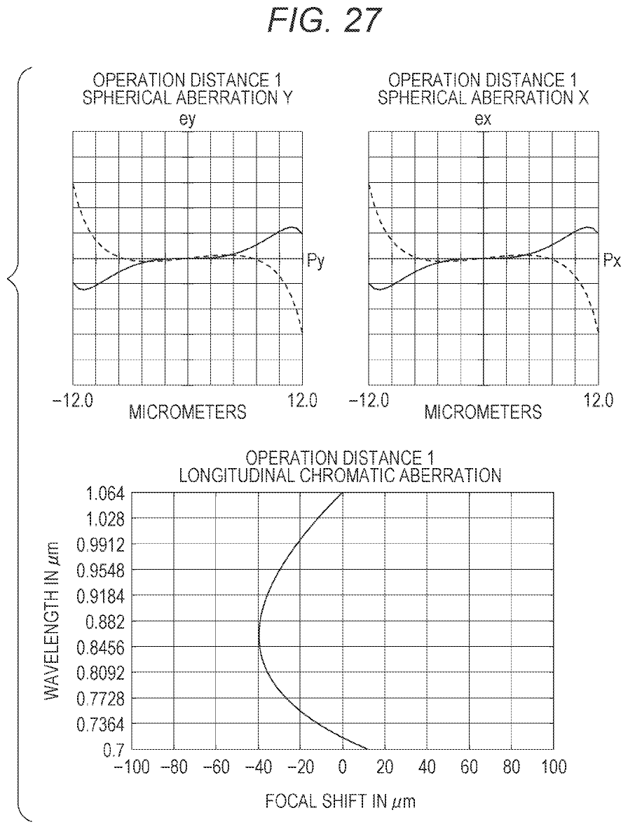

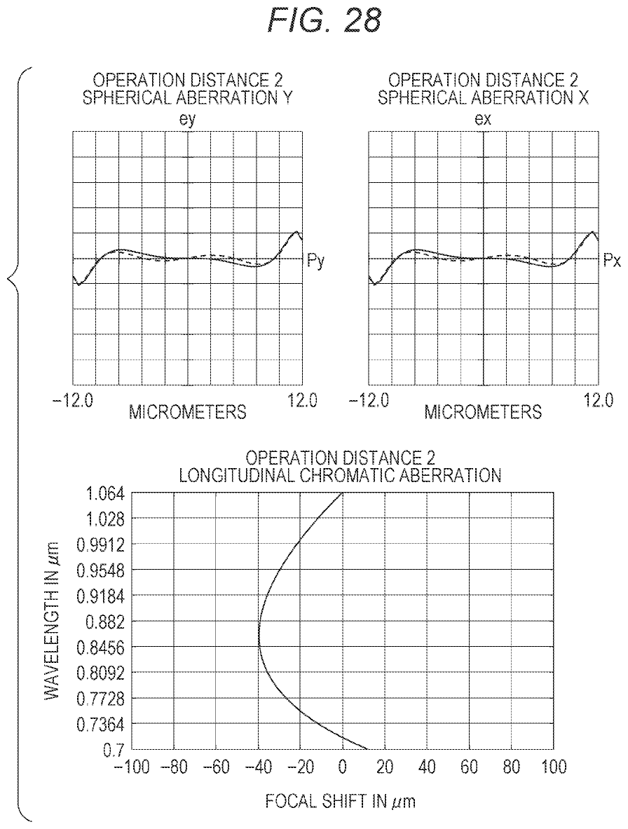

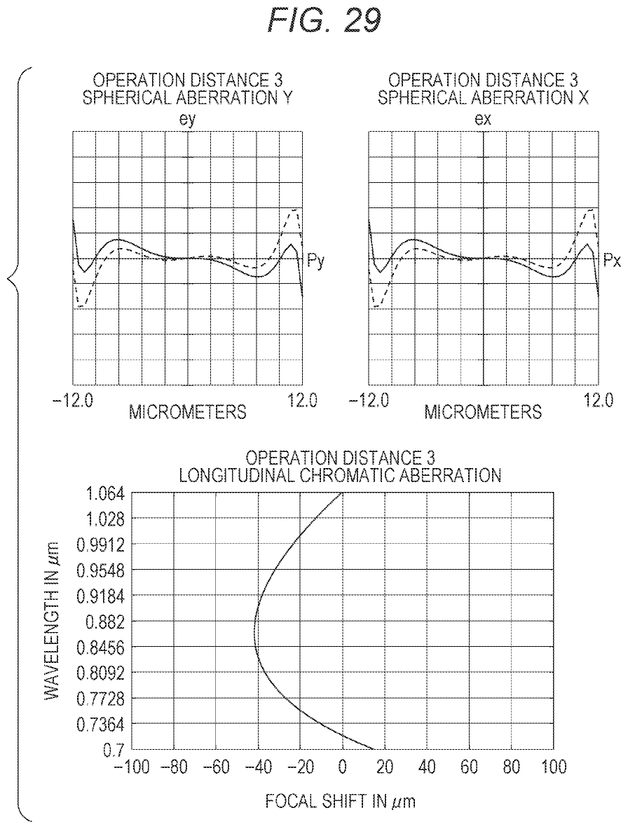

FIG. 26 schematically illustrates an optical configuration of the condensing objective optical system of Example 7, and FIGS. 27 to 29 illustrate graphs of spherical aberration and graphs of longitudinal chromatic aberration at the operation distances 1 to 3 in Example 7. Table 13 shows the optical data in Example 7, and Table 14 shows the object distance in Example 7, and the focal distance, the F number (FNo), the distance between the lens groups, and the distance between the lens and the prism of the condensing objective optical system corresponding thereto.

TABLE 13

| Face | | | | | |
|---|---|---|---|---|---|
| number | | R | D | N | ν |
| 1 | STO | 0.000 | 10.000 | | |
| 2 | | −10.487 | 4.400 | 1.946 | 17.984 |
| 3 | | −19.799 | 10.422 | | |
| 4 | | 168.218 | 3.000 | 1.835 | 42.721 |
| 5 | | −32.890 | (1) | | |
| 6 | | 3217.373 | 1.100 | 1.497 | 81.608 |
| 7 | | 13.301 | 3.535 | | |
| 8 | | 14.909 | 1.000 | 1.805 | 25.456 |
| 9 | | 11.575 | 5.000 | 1.497 | 81.608 |
| 10 | | −26.222 | (2) | | |
| 11 | P | 0.000 | 15.000 | 1.911 | 35.250 |
| 12 | | 0.000 | (3) | 1.333 | 55.794 |

TABLE 14

| | Operation | Operation | Operation |
|---|---|---|---|
| | distance 1 | distance 2 | distance 3 |
| Focal distance | 32.521 | 30.309 | 29.338 |
| FNo | 3.613 | 3.368 | 3.260 |
| (1) | 22.628 | 13.000 | 8.313 |
| (2) | 10.917 | 20.546 | 5.233 |
| (3) | 30.000 | 20.000 | 15.000 |

### Example 8

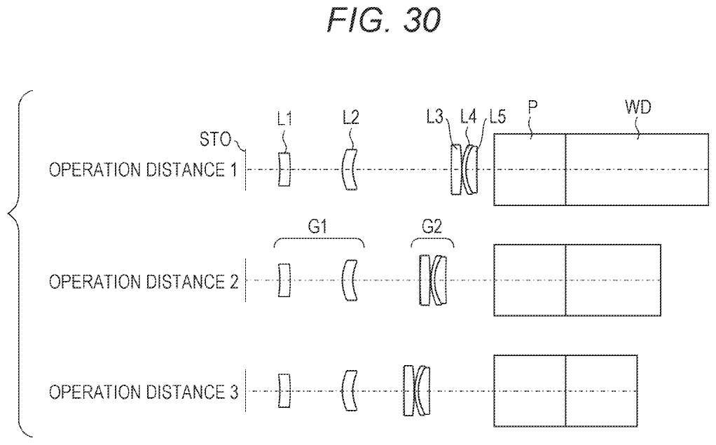

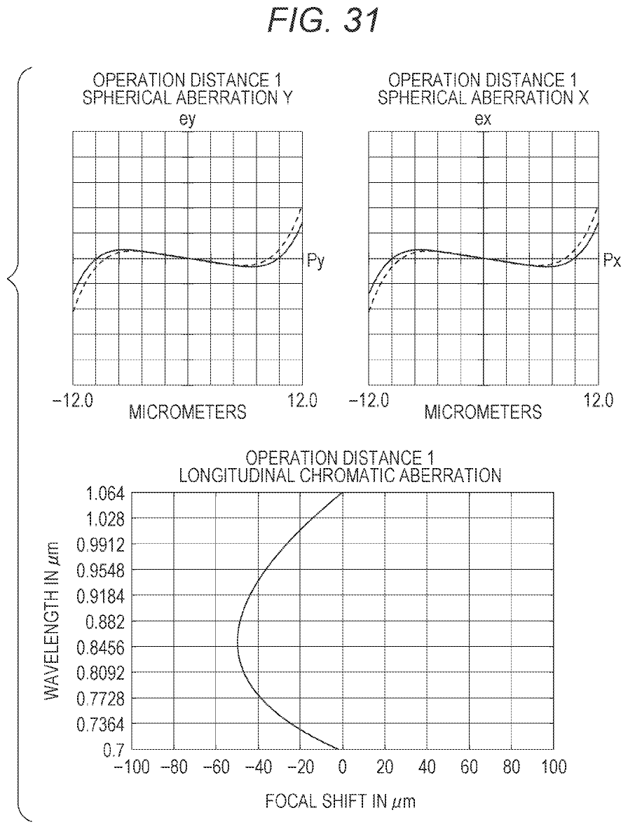

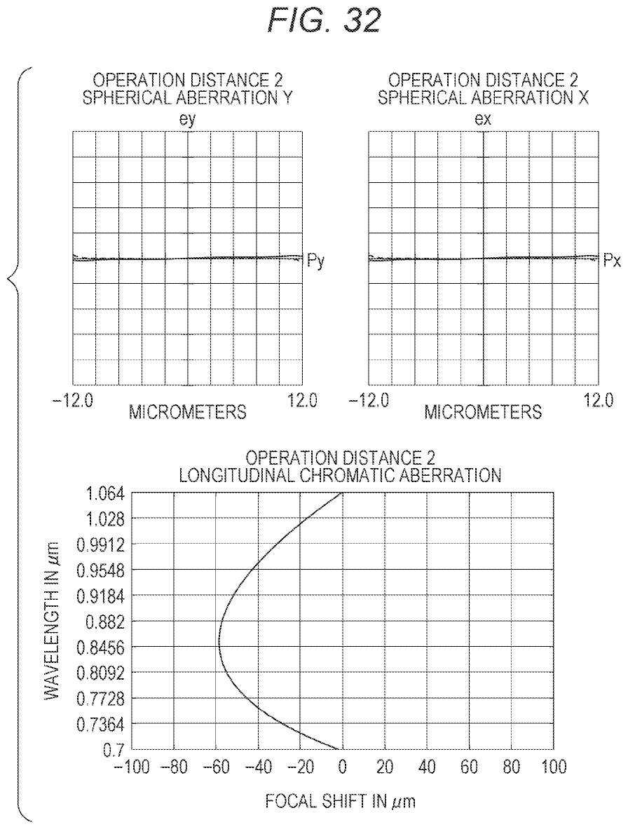

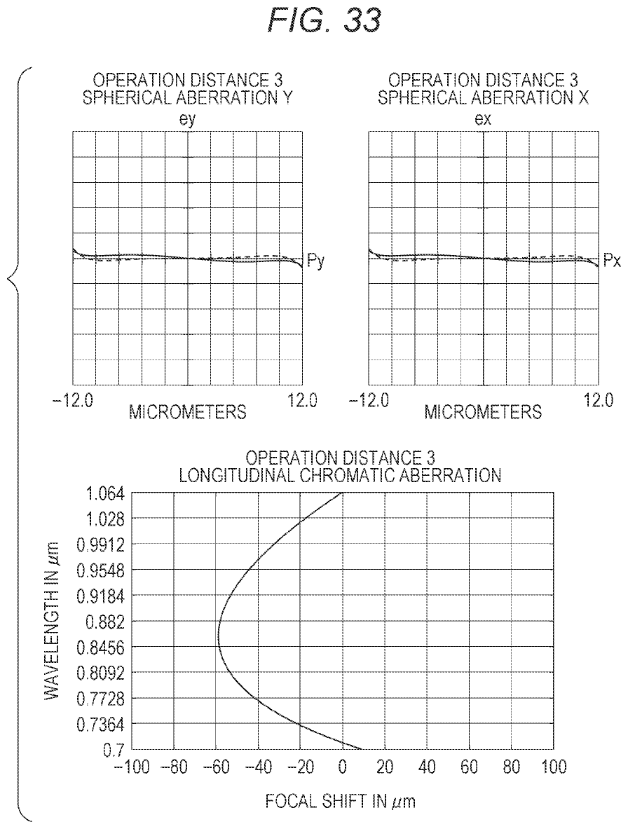

FIG. 30 schematically illustrates an optical configuration of the condensing objective optical system of Example 8, and FIGS. 31 to 33 illustrate graphs of spherical aberration and graphs of longitudinal chromatic aberration at the operation distances 1 to 3 in Example 8. Table 15 shows the optical data in Example 8, and Table 16 shows the object distance in Example 8, and the focal distance, the F number (FNo), the distance between the lens groups, and the distance between the lens and the prism of the condensing objective optical system corresponding thereto.

TABLE 15

| Face | | | | | |
|---|---|---|---|---|---|
| number | | R | D | N | ν |
| 1 | STO | 0.000 | 10.000 | | |
| 2 | | −28.495 | 3.000 | 1.946 | 17.984 |
| 3 | | −51.365 | 15.055 | | |
| 4 | | 13.254 | 3.000 | 1.954 | 32.319 |
| 5 | | 11.739 | (1) | | |
| 6 | | 164.747 | 3.000 | 1.804 | 46.503 |
| 7 | | −70.873 | 0.200 | | |
| 8 | | 16.707 | 1.000 | 1.923 | 20.880 |
| 9 | | 12.563 | 3.200 | 1.487 | 70.441 |
| 10 | | 185.467 | (2) | | |
| 11 | P | 0.000 | 15.000 | 1.911 | 35.250 |
| 12 | | 0.000 | (3) | 1.333 | 55.794 |

TABLE 16

| | Operation | Operation | Operation |
|---|---|---|---|
| | distance 1 | distance 2 | distance 3 |
| Focal distance | 24.815 | 28.209 | 30.382 |
| FNo | 2.757 | 3.134 | 3.376 |
| (1) | 28.546 | 19.518 | 14.797 |
| (2) | 4.999 | 14.028 | 18.748 |
| (3) | 30.000 | 20.000 | 15.000 |

### Example 9

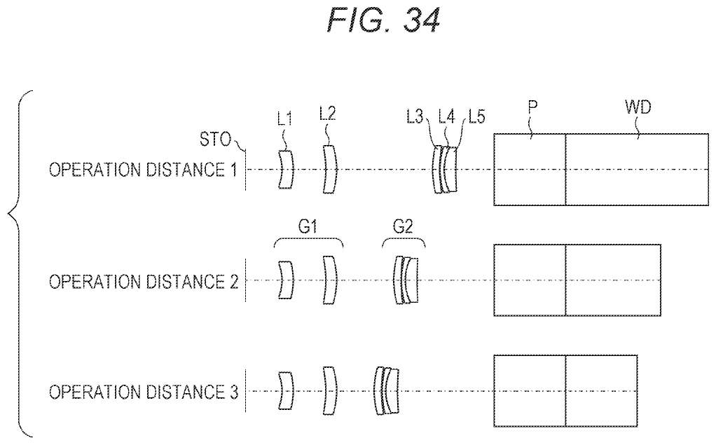

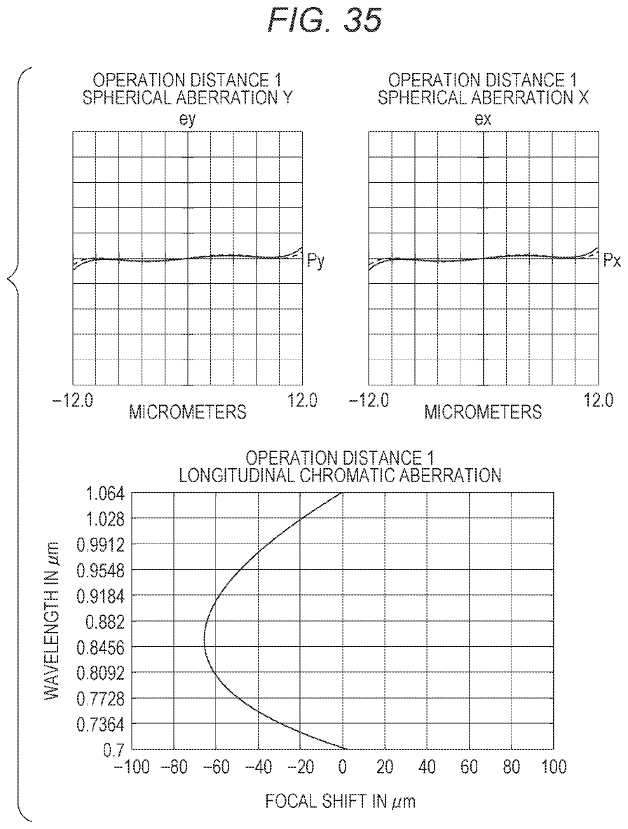

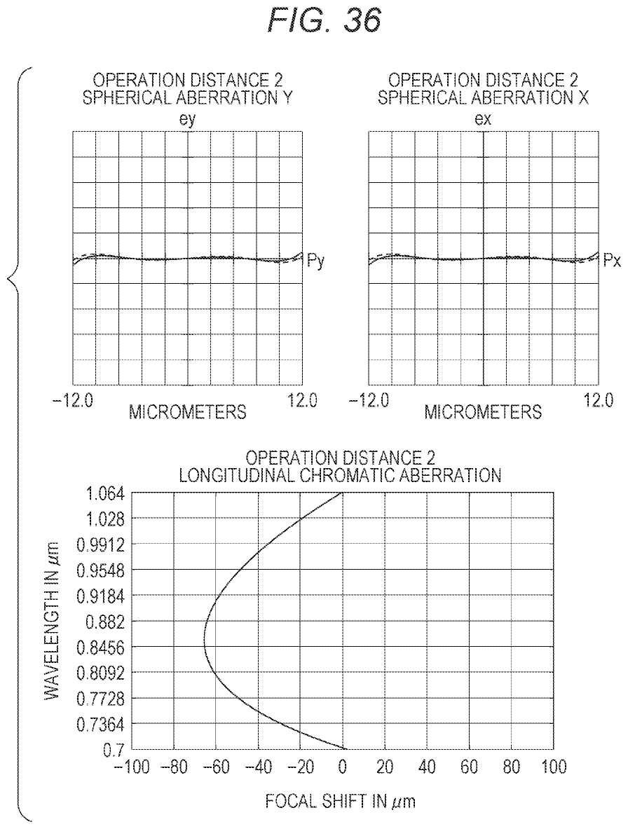

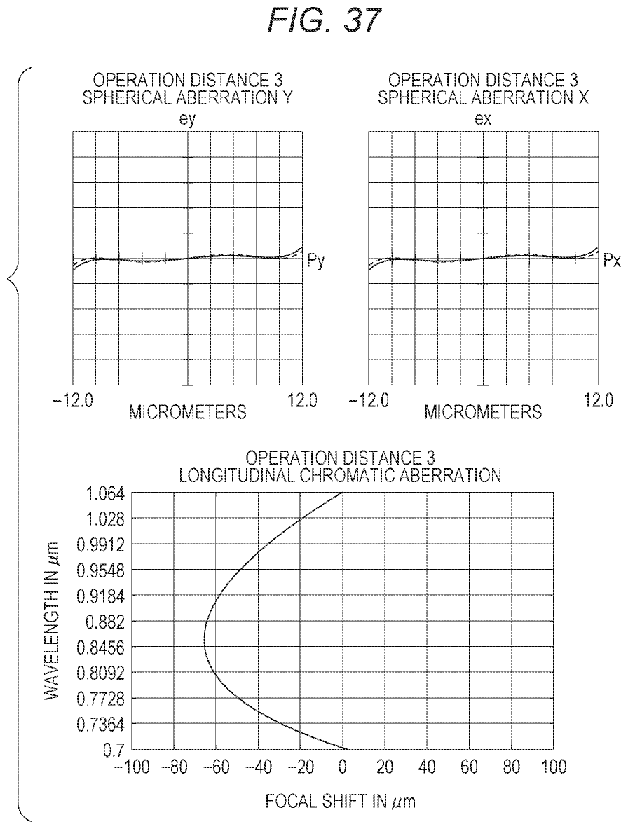

FIG. 34 schematically illustrates an optical configuration of the condensing objective optical system of Example 9, and FIGS. 35 to 37 illustrate graphs of spherical aberration and graphs of longitudinal chromatic aberration at the operation distances 1 to 3 in Example 9. Table 17 shows the optical data in Example 9, and Table 18 shows the object distance in Example 9, and the focal distance, the F number (FNo), the distance between the lens groups, and the distance between the lens and the prism of the condensing objective optical system corresponding thereto.

TABLE 17

| Face | | | | | |
|---|---|---|---|---|---|
| number | | R | D | N | ν |
| 1 | STO | 0.000 | 10.000 | | |
| 2 | | 12.579 | 3.000 | 1.946 | 17.984 |
| 3 | | −20.399 | 9.000 | | |
| 4 | | −28.639 | 3.000 | 1.954 | 32.319 |
| 5 | | −20.185 | (1) | | |
| 6 | | 23.853 | 2.000 | 1.773 | 49.624 |
| 7 | | 37.065 | 0.200 | | |
| 8 | | 23.534 | 1.000 | 1.805 | 25.456 |
| 9 | | 13.285 | 3.200 | 1.517 | 64.198 |
| 10 | | 80.632 | (2) | | |
| 11 | P | 0.000 | 15.000 | 1.911 | 35.250 |
| 12 | | 0.000 | (3) | 1.000 | 0.000 |

TABLE 18

| | Operation | Operation | Operation |
|---|---|---|---|
| | distance 1 | distance 2 | distance 3 |
| Focal distance | 36.748 | 36.748 | 36.748 |
| FNo | 4.083 | 4.083 | 4.083 |
| (1) | 26.141 | 15.542 | 10.249 |
| (2) | 7.405 | 18.003 | 23.297 |
| (3) | 30.594 | 20.000 | 14.703 |

### Example 10

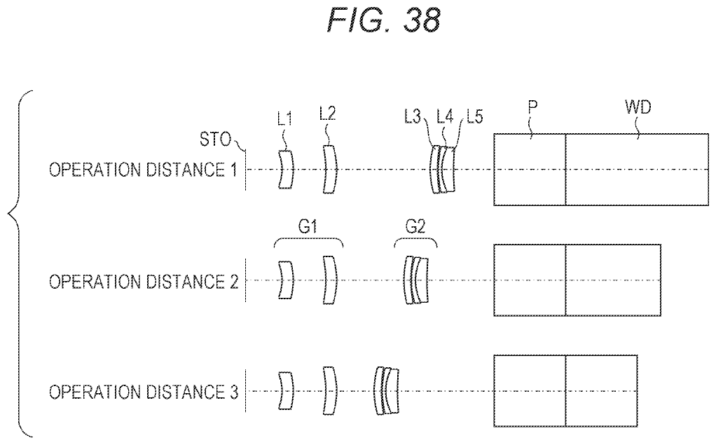

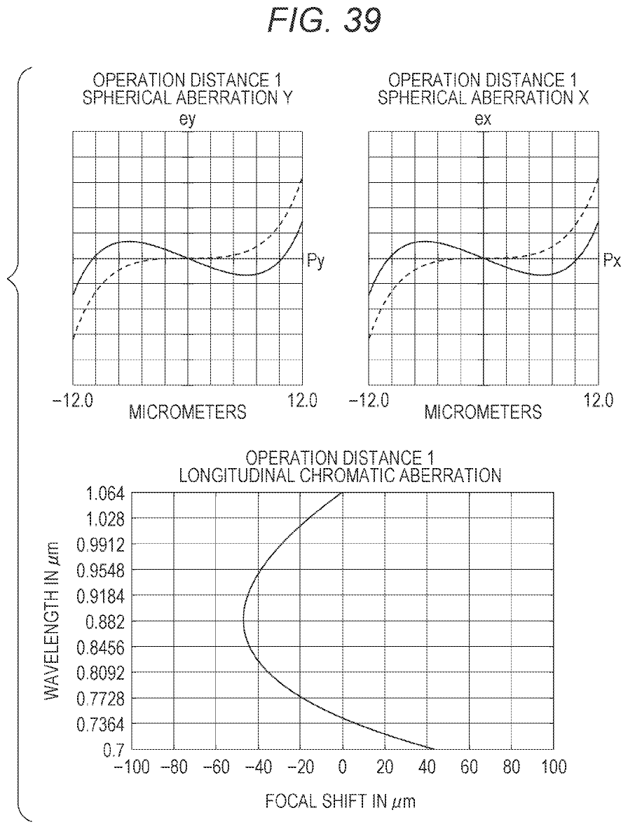

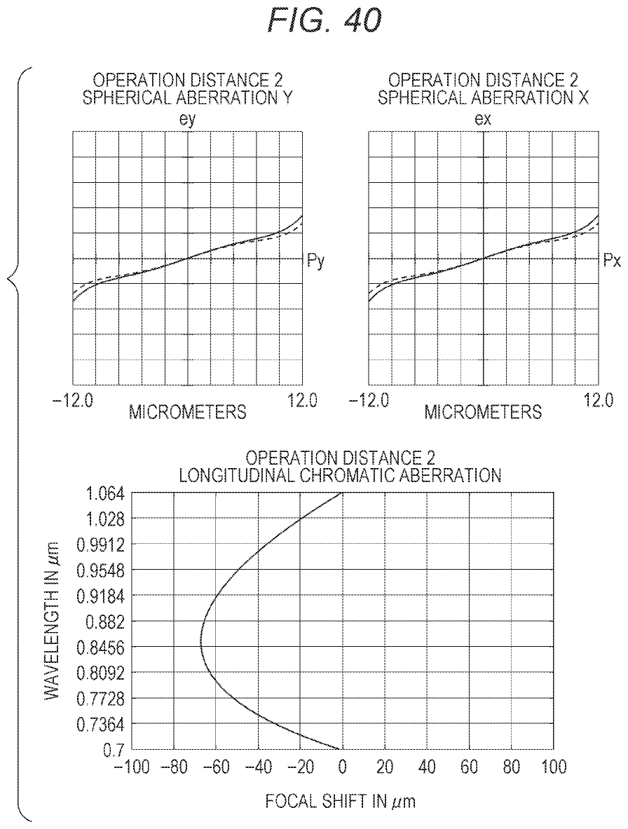

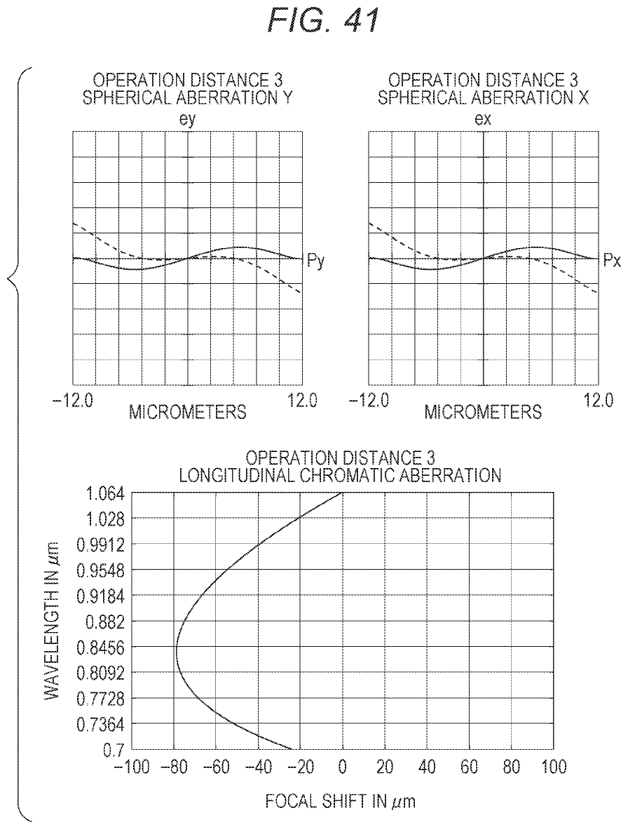

FIG. 38 schematically illustrates the optical configuration of the condensing objective optical system of Example 10, and FIGS. 39 to 41 illustrate graphs of spherical aberration and graphs of longitudinal chromatic aberration at the operation distances 1 to 3 in Example 10. Table 19 shows the optical data in Example 10, and Table 20 shows the object distance in Example 10, and the focal distance, the F number (FNo), the distance between the lens groups, and the distance between the lens and the prism of the condensing objective optical system corresponding thereto.

TABLE 19

| Face | | | | | |
|---|---|---|---|---|---|
| number | | R | D | N | ν |
| 1 | STO | 0.000 | 10.000 | | |
| 2 | | −12.579 | 3.000 | 1.946 | 17.984 |
| 3 | | −20.399 | 9.000 | | |
| 4 | | −28.639 | 3.000 | 1.954 | 32.319 |
| 5 | | −20.185 | (1) | | |
| 7 | | 23.650 | 2.000 | 1.773 | 49.624 |
| 8 | | 39.361 | 0.200 | | |
| 9 | | 23.420 | 1.000 | 1.805 | 25.456 |
| 10 | | 13.002 | 3.200 | 1.517 | 64.198 |
| 11 | | 252.342 | (2) | | |
| 12 | P | 0.000 | 15.000 | 1.911 | 35.250 |
| 13 | | 0.000 | (3) | 1.517 | 64.198 |

TABLE 20

| | Operation | Operation | Operation |
|---|---|---|---|
| | distance 1 | distance 2 | distance 3 |
| Focal distance | 29.205 | 29.205 | 29.205 |
| FNo | 3.245 | 3.245 | 3.245 |
| (1) | 25.633 | 18.569 | 15.038 |
| (2) | 7.912 | 14.977 | 18.507 |
| (3) | 30.924 | 20.200 | 14.883 |

### Example 11

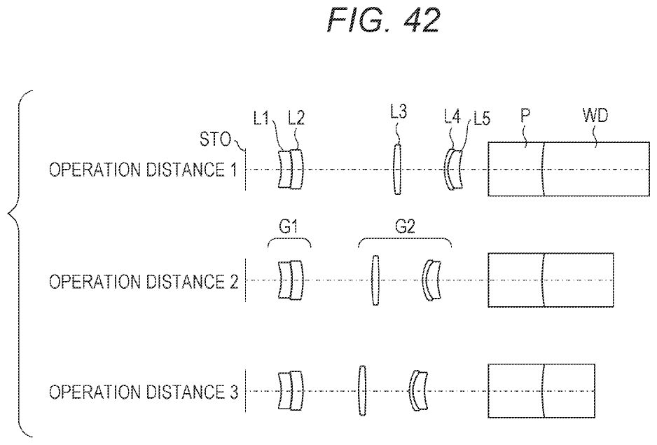

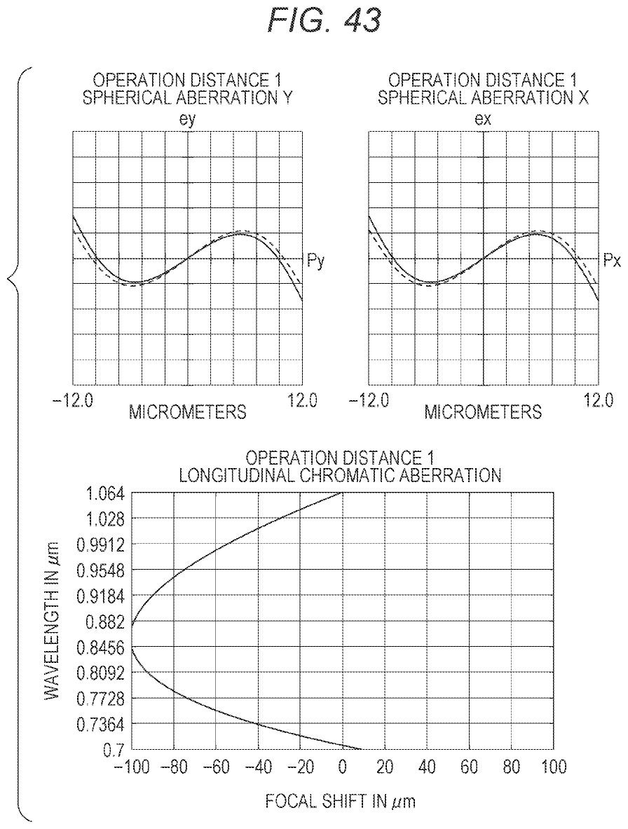

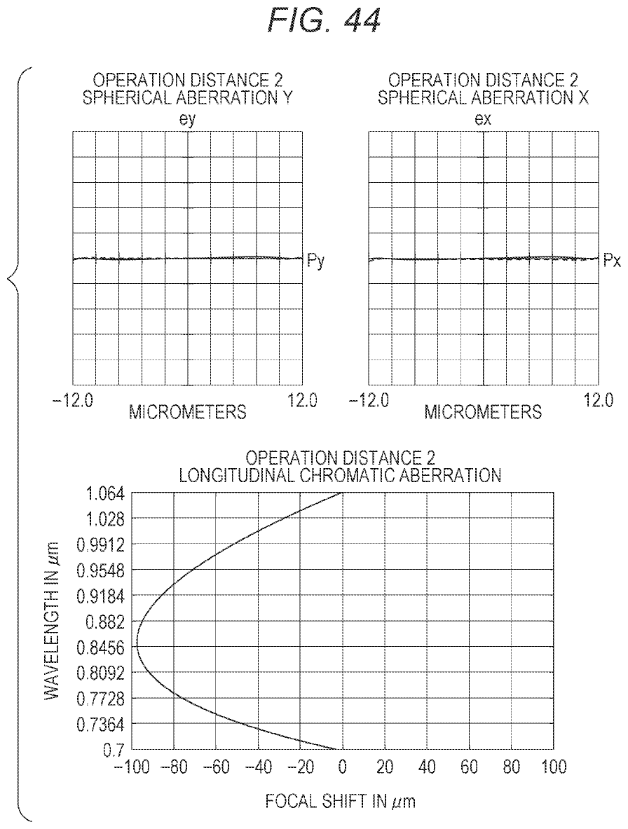

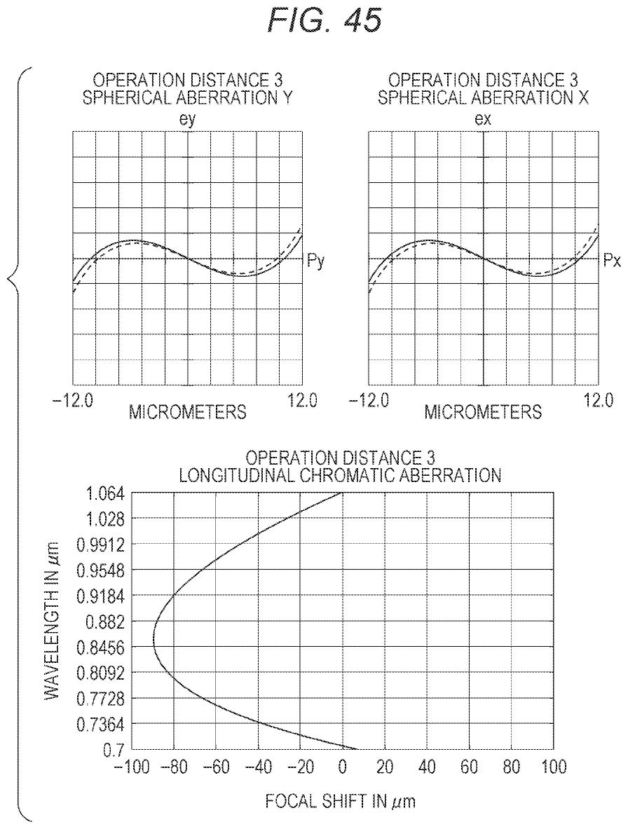

FIG. 42 schematically illustrates the optical configuration of the condensing objective optical system of Example 11, and FIGS. 43 to 45 illustrate graphs of spherical aberration and graphs of longitudinal chromatic aberration at the operation distances 1 to 3 in Example 11. Table 21 shows the optical data in Example 11, and Table 22 shows the object distance in Example 11, and the focal distance, the F number (FNo), the distance between the lens groups, and the distance between the lens and the prism of the condensing objective optical system corresponding thereto. As illustrated in FIG. 42, the prism P in the present Example faces the portion of the object distance with a paraboloid concave curved face.

TABLE 21

| Face | | | | | |
|---|---|---|---|---|---|
| number | | R | D | N | ν |
| 1 | STO | 0.000 | 10.000 | | |
| 2 | | −14.758 | 3.000 | 1.946 | 17.984 |
| 3 | | −25.455 | 0.100 | | |
| 4 | | −29.414 | 3.000 | 1.954 | 32.319 |
| 5 | | −24.091 | (1) | | |
| 7 | | 48.750 | 2.000 | 1.773 | 49.624 |
| 8 | | −146.363 | 12.251 | | |
| 9 | | 11.576 | 1.000 | 1.805 | 25.456 |
| 10 | | 8.554 | 3.200 | 1.517 | 64.198 |
| 11 | | 15.146 | (2) | | |
| 12 | P | 0.000 | 15.000 | 1.911 | 35.250 |
| 13 | | 0.000 | (3) | 1.333 | 55.794 |

TABLE 22

| | Operation | Operation | Operation |
|---|---|---|---|
| | distance 1 | distance 2 | distance 3 |
| Focal distance | 41.793 | 41.588 | 41.683 |
| FNo | 4.464 | 4.621 | 4.631 |
| (1) | 25.370 | 19.280 | 15.590 |
| (2) | 8.175 | 14.266 | 17.956 |
| (3) | 30.000 | 20.000 | 14.700 |

### Example 12

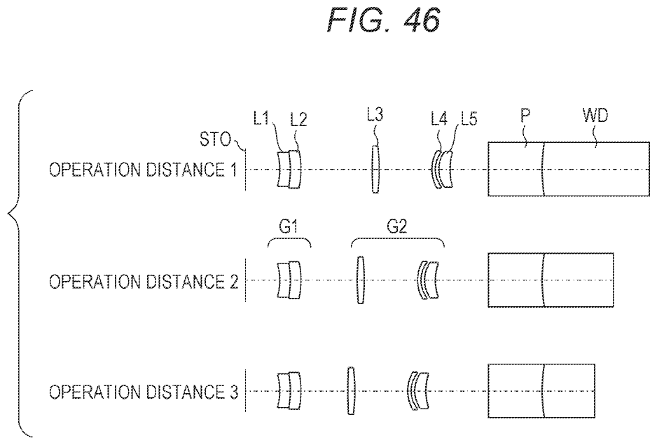

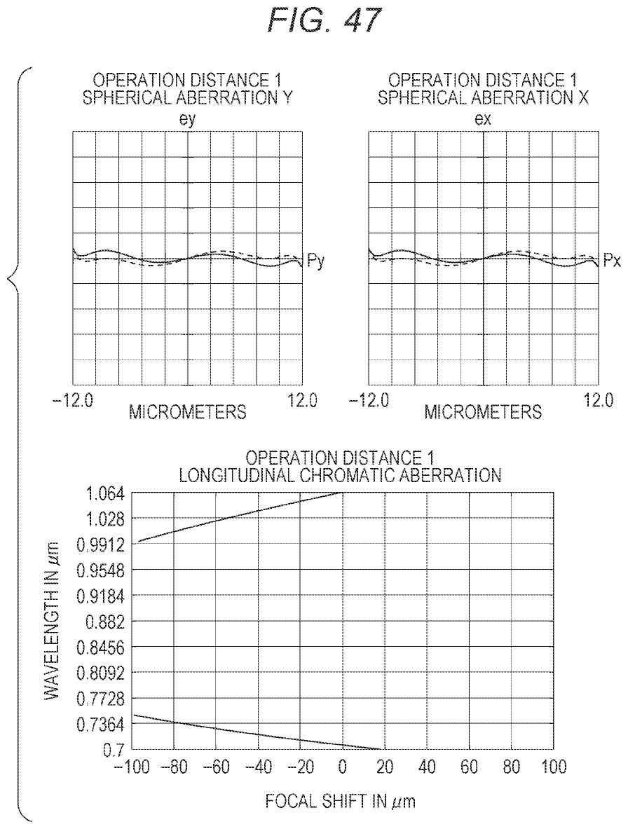

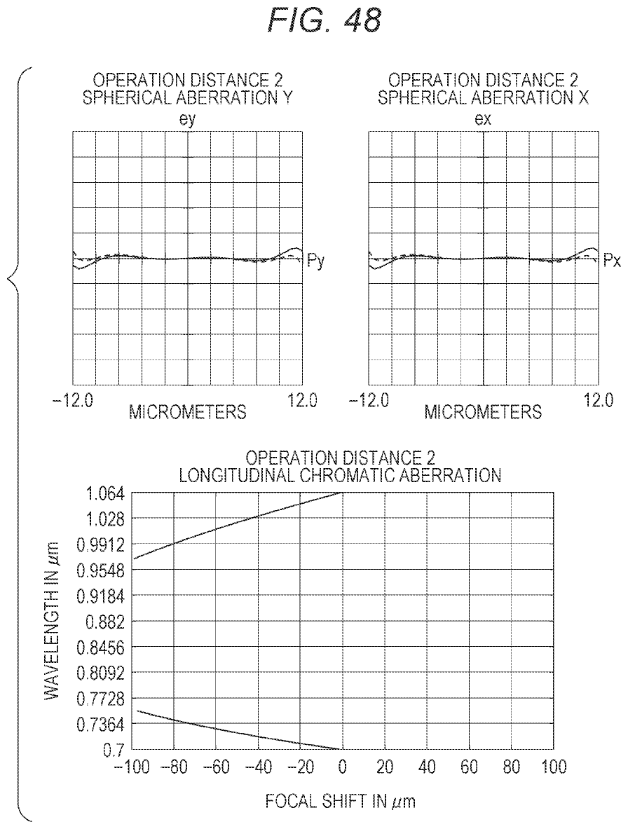

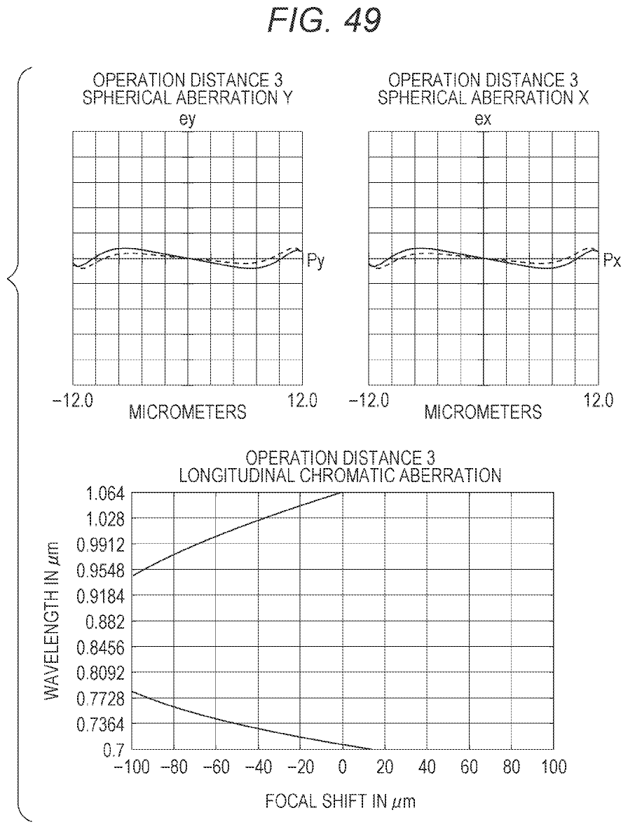

FIG. 46 schematically illustrates the optical configuration of the condensing objective optical system of Example 12, and FIGS. 47 to 49 illustrate graphs of spherical aberration and graphs of longitudinal chromatic aberration at the operation distances 1 to 3 in Example 12. Table 23 shows the optical data in Example 12, and Table 24 shows the object distance in Example 12, and the focal distance, the F number (FNo), the distance between the lens groups, and the distance between the lens and the prism of the condensing objective optical system corresponding thereto. As illustrated in FIG. 46, the prism P in the present example faces the portion WD of the object distance with a paraboloid concave curved face.

TABLE 23

| Face | | | | | |
|---|---|---|---|---|---|
| number | | R | D | N | ν |
| 1 | STO | 0.000 | 10.000 | | |
| 2 | | −16.235 | 3.000 | 1.946 | 17.981 |
| 3 | | −29.358 | 0.100 | | |
| 4 | | −25.243 | 3.000 | 1.954 | 32.319 |
| 5 | | −22.662 | (1) | | |
| 7 | | 88.733 | 2.000 | 1.773 | 49.624 |
| 8 | | −61.959 | 15.425 | | |
| 9 | | 11.151 | 1.000 | 1.805 | 25.456 |
| 10 | | 8.422 | 1.000 | | |
| 11 | | 8.713 | 3.200 | 1.517 | 64.198 |
| 12 | | 15.930 | (2) | | |
| 13 | P | 0.000 | 15.000 | 1.911 | 35.250 |
| 14 | | 0.000 | (3) | 1.333 | 55.794 |

TABLE 24

| | Operation | Operation | Operation |
|---|---|---|---|
| | distance 1 | distance 2 | distance 3 |
| Focal distance | 55.390 | 51.006 | 48.901 |
| FNo | 6.154 | 5.667 | 5.433 |
| (1) | 20.664 | 16.474 | 13.607 |
| (2) | 12.881 | 17.072 | 19.938 |
| (3) | 30.000 | 20.000 | 14.700 |

### Example 13

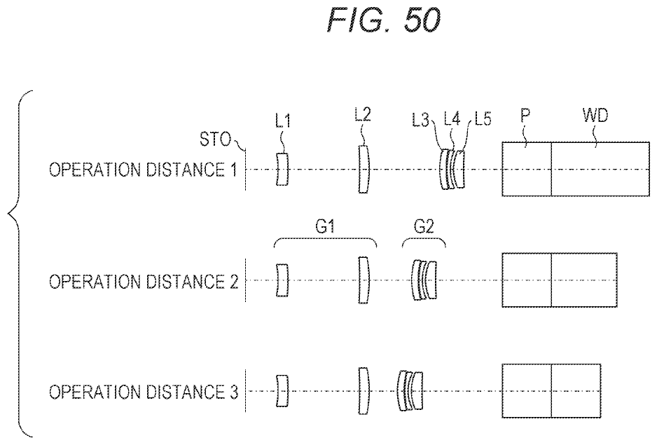

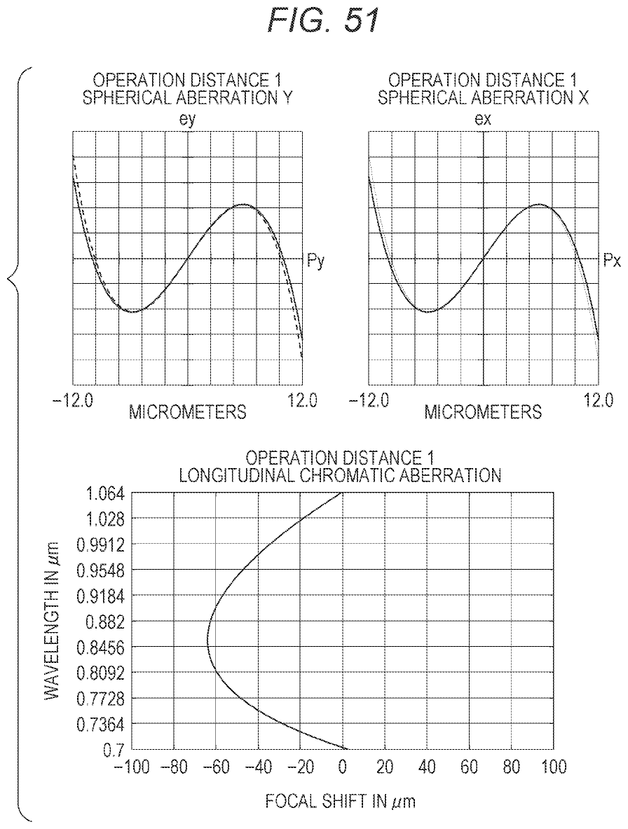

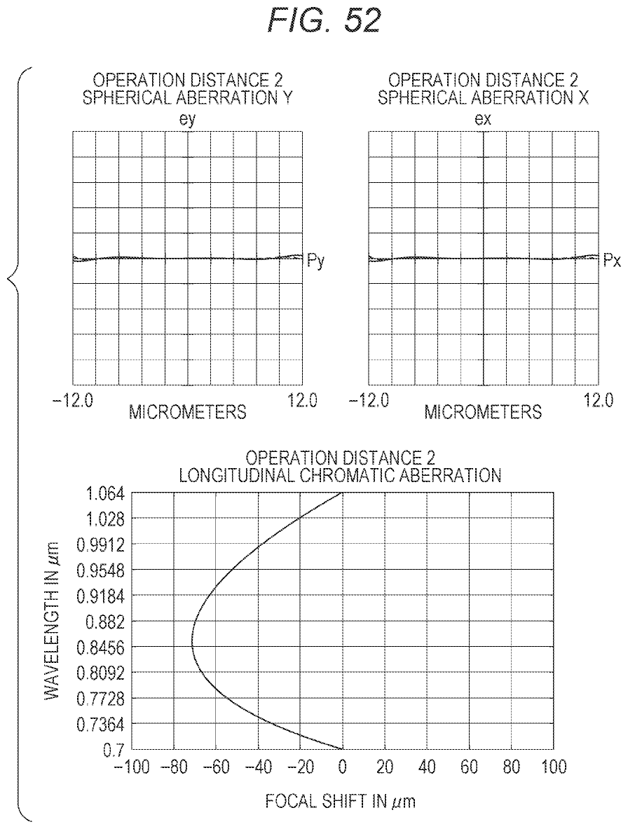

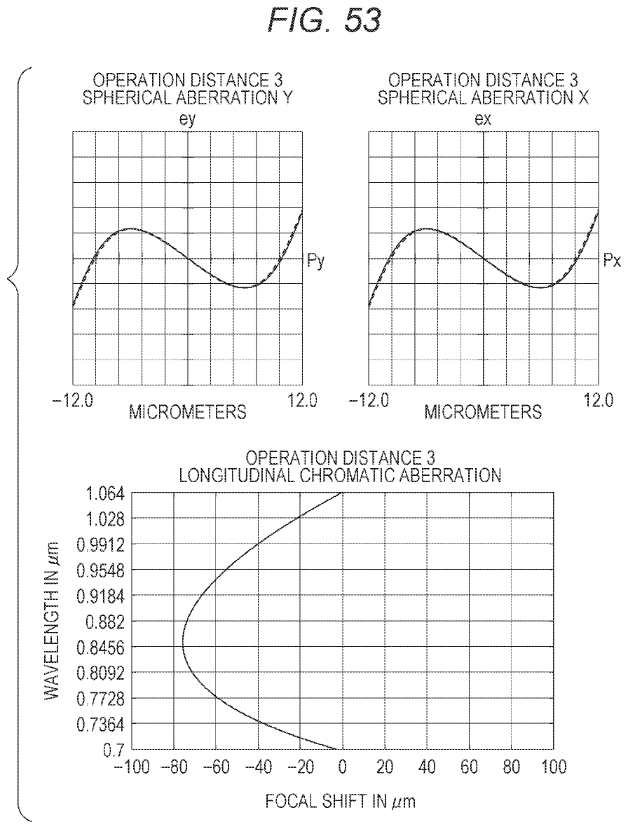

FIG. 50 schematically illustrates the optical configuration of the condensing objective optical system of Example 13, and FIGS. 51 to 53 illustrate graphs of spherical aberration and graphs of longitudinal chromatic aberration at the operation distances 1 to 3 in Example 13. Table 25 shows the optical data in Example 13, and Table 26 shows the object distance in Example 13, and the focal distance, the F number (FNo), the distance between the lens groups, and the distance between the lens and the prism of the condensing objective optical system corresponding thereto.

TABLE 25

| Face | | | | | |
|---|---|---|---|---|---|
| number | | R | D | N | ν |
| 1 | STO | 0.000 | 10.000 | | |
| 2 | | −24.458 | 3.000 | 1.946 | 17.984 |
| 3 | | −58.878 | 22.070 | | |
| 4 | | −105.458 | 3.000 | 1.954 | 32.319 |
| 5 | | −38.519 | (1) | | |
| 6 | | 21.212 | 2.000 | 1.773 | 49.624 |
| 7 | | 25.732 | 0.200 | | |
| 8 | | 24.228 | 1.000 | 1.805 | 25.456 |
| 9 | | 12.578 | 1.000 | | |
| 10 | | 13.034 | 3.200 | 1.517 | 64.198 |
| 11 | | 111.044 | (2) | | |
| 12 | P | 0.000 | 15.000 | 1.729 | 54.674 |
| 13 | | 0.000 | (3) | 1.333 | 55.794 |

TABLE 26

| | Operation | Operation | Operation |
|---|---|---|---|
| | distance 1 | distance 2 | distance 3 |
| Focal distance | 36.101 | 34.753 | 34.117 |
| FNo | 4.011 | 3.861 | 3.791 |
| (1) | 21.612 | 12.999 | 8.698 |
| (2) | 11.934 | 20.546 | 24.847 |
| (3) | 30.000 | 20.000 | 15.000 |

### Example 14

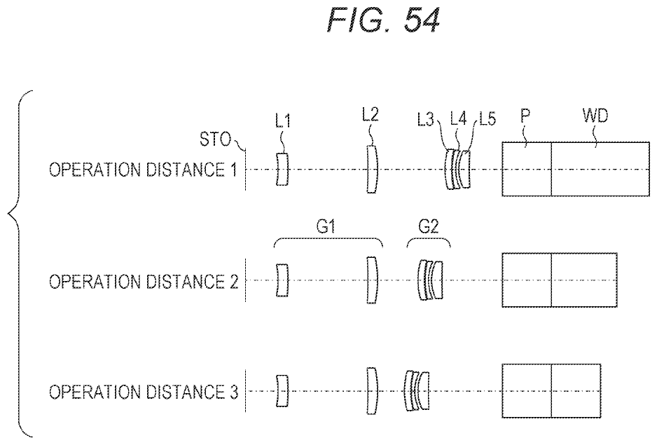

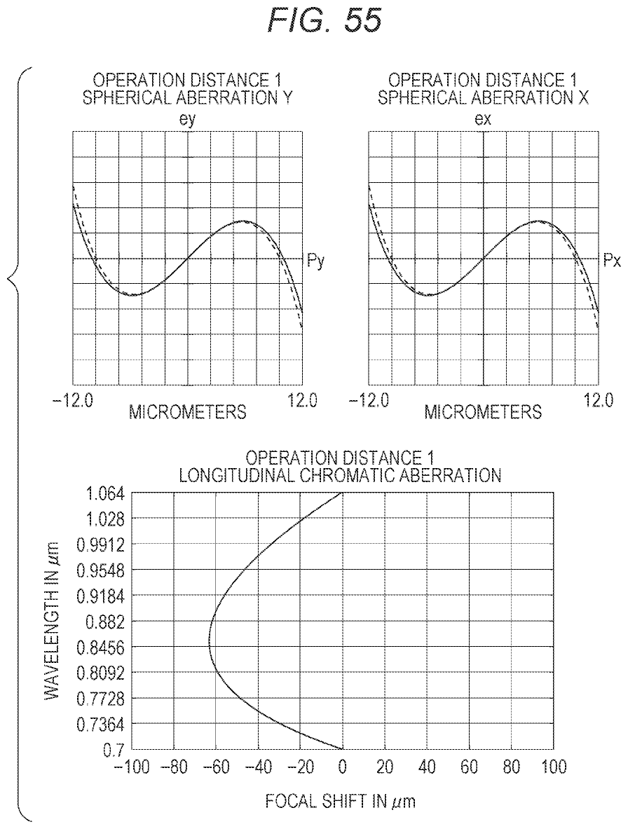

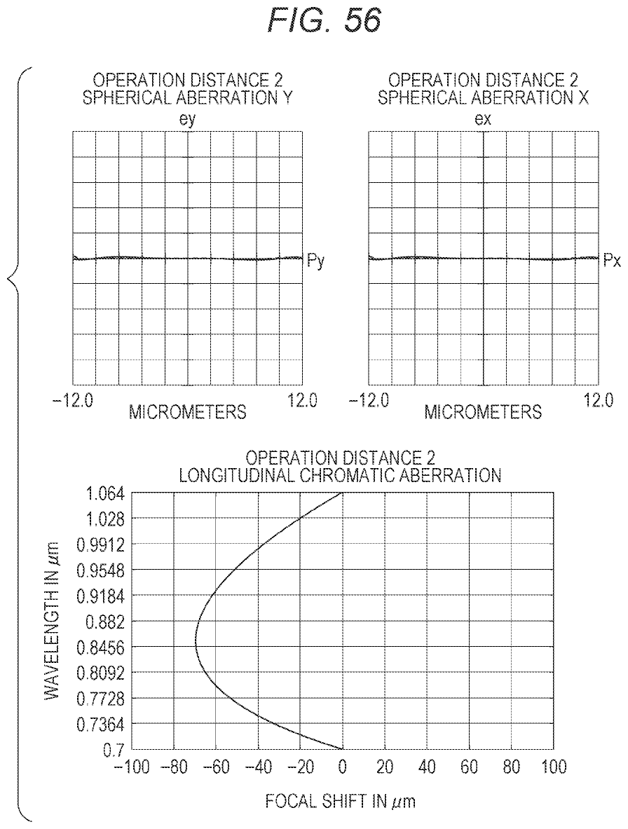

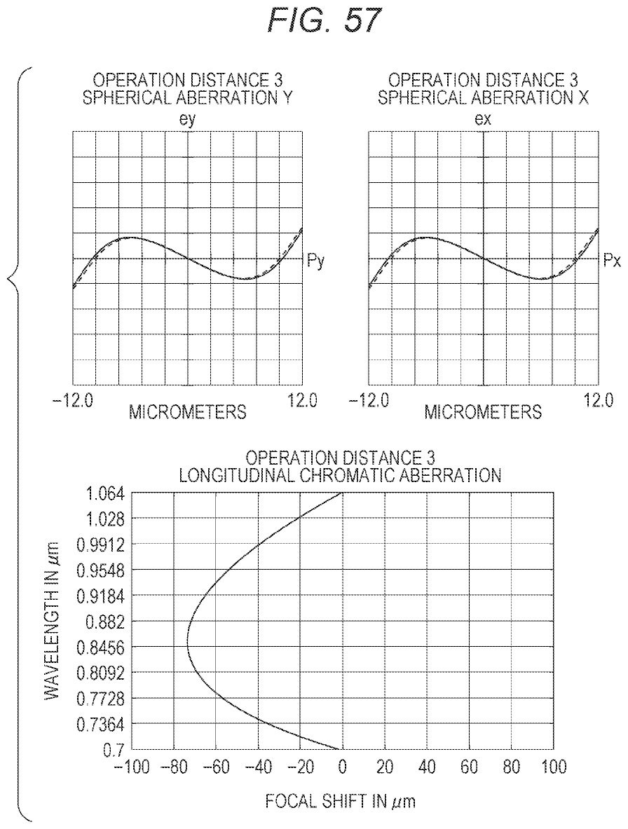

FIG. 54 schematically illustrates the optical configuration of the condensing objective optical system of Example 14, and FIGS. 55 to 57 illustrate graphs of spherical aberration and graphs of longitudinal chromatic aberration at the operation distances 1 to 3 in Example 14. Table 27 shows the optical data in Example 14, and Table 28 shows the object distance in Example 14, and the focal distance, the F number (FNo), the distance between the lens groups, and the distance between the lens and the prism of the condensing objective optical system corresponding thereto.

TABLE 27

| Face | | | | | |
|---|---|---|---|---|---|
| number | | R | D | N | ν |
| 1 | STO | 0.000 | 10.000 | | |
| 2 | | 0.000 | 3.000 | 1.946 | 17.984 |
| 3 | | −57.381 | 5.606 | | |
| 4 | | −139.562 | 3.000 | 1.954 | 32.319 |
| 5 | | −37.993 | (1) | | |
| 6 | | 20.836 | 2.000 | 1.773 | 49.624 |
| 7 | | 26.299 | 0.200 | | |
| 8 | | 24.230 | 1.000 | 1.805 | 25.456 |
| 9 | | 12.578 | 1.000 | | |
| 10 | | 12.952 | 3.200 | 1.517 | 64.198 |
| 11 | | 82.587 | (2) | | |
| 12 | P | 0.000 | 15.000 | 2.051 | 26.942 |
| 13 | | 0.000 | (3) | 1.333 | 55.794 |

TABLE 28

| | Operation | Operation | Operation |
|---|---|---|---|
| | distance 1 | distance 2 | distance 3 |
| Focal distance | 35.493 | 34.122 | 33.480 |
| FNo | 3.943 | 3.791 | 3.720 |
| (1) | 21.632 | 12.999 | 8.718 |
| (2) | 11.914 | 20.546 | 24.827 |
| (3) | 30.000 | 20.000 | 15.000 |

A list of values according to the expressions in each of the above Examples is shown in the following table.

TABLE 29

| | Example | Example | Example | Example |
|---|---|---|---|---|
| | 1 | 2 | 3 | 4 |
| Expression (1) vd | 70.44, | 75.5, | 81.61 | 70.44 |
| | 95.10 | 81.61 | | |
| Expression (2) f/fvMAX | 0.294 | 0.407 | 1.191 | 1.648 |
| Expression (3) ΔZ/Lf | 1.270 | 1.318 | 1.308 | 1.315 |
| Expression (4) a12 | 1.262 | 0.440 | 0.701 | 0.480 |
| Expression (5) nWD | 1.333 | 1.333 | 1.333 | 1.333 |
| Expression (6) np | 1.911 | 1.911 | 1.911 | 1.911 |

TABLE 30

| | | Example 5 | Example 6 | Example 7 | Example 8 |
|---|---|---|---|---|---|
| Expression (1) | νd | 70.44 | 64.20 | 81.61 | 70.44 |
| Expression (2) | | 1.648 | 1.080 | 1.89 7 | 1.085 |
| f/fνMAX | | | | | |
| Expression (3) | ΔZ/Lf | 1.315 | 1.330 | 1.040 | 1.108 |
| Expression (4) | a12 | 0.480 | 0.000 | −4.000 | 4.000 |
| Expression (5) | nWD | 1.333 | 1.333 | 1.333 | 1.333 |
| Expression (6) | np | 1.911 | 1.911 | 1.911 | 1.911 |

TABLE 31

| | | Example | Example | Example | Example |
|---|---|---|---|---|---|
| | | 9 | 10 | 11 | 12 |
| Expression (1) | νd | 64.20 | 64.20 | 64.20 | 64.20 |
| Expression (2) | | 1.210 | 1.084 | 1.253 | 1.265 |
| f/fνMAX | | | | | |
| Expression (3) | ΔZ/Lf | 1.108 | 1.518 | 1.642 | 2.386 |
| Expression (4) | a12 | 0.000 | 0.000 | 3.070 | 3.650 |
| Expression (5) | nWD | 1.000 | 1.517 | 1.333 | 1.333 |
| Expression (6) | np | 1.911 | 1.911 | 1.911 | 1.911 |

TABLE 32

| | Example 13 | Example 14 |
|---|---|---|
| Expression (1) vd | 64.20 | 64.20 |
| Expression (2) f/fvMAX | 1.450 | 1.400 |
| Expression (3) ΔZ/Lf | 1.161 | 1.159 |
| Expression (4) a12 | 2.520 | 2.610 |
| Expression (5) nWD | 1.333 | 1.333 |
| Expression (6) np | 1.729 | 2.051 |

What is claimed is:

1. A condensing objective optical system comprising:
a first lens group having negative refractive power;
a second lens group having positive refractive power, the second lens group being movable along an optical axis so as to change a distance between the second lens group and an adjacent lens group; and
a photoacoustic element that is disposed closest to an object, configured to reflect an optical wave incident from the second lens group toward the object, and configured to transmit a photoacoustic wave emitted by the object that absorbed the optical wave, in this order, wherein
the condensing objective optical system includes at least one lens satisfying following Expressions (1) and (2), and satisfies following Expression (3):
$$\begin{matrix}vd > 64 & ( 1 ) \\ 0.294 \leq f \text{/} fvMAX < 2.140 & ( 2 ) \\ 1.000 < Δ Z \text{/} Lf \leq 2.386 & ( 3 )\end{matrix}$$
where
νd is an Abbe number with respect to d Line,
f is a focal distance when an operation distance of the condensing objective optical system is longest,
fνMAX is a focal distance of a lens the νd of which is maximum,
ΔZ is an amount of change in a distance on an optical axis from a surface, of the photoacoustic element, on the object side to an imaging position at the object side in the condensing objective optical system, and
Lf is a movement distance of the second lens group.

2. The condensing objective optical system according to claim 1, wherein
when a direction in which a pencil of light of the optical wave diverges is positive and a direction in which the pencil of light condenses is negative, following Expression (4) is satisfied:
$$- 4.0 ° \leq a 12 \leq 4.0 ° \qquad ( 4 )$$
where
a12 is a divergence angle of the pencil of light of the optical wave between the first lens group and the second lens group.

3. The condensing objective optical system according to claim 1, wherein
the condensing objective optical system is configured based on a portion through which the optical wave from the photoacoustic element to the object passes, the portion satisfying following Expression (5):
$$nWD < 1.53 \qquad ( 5 )$$
where
nWD is a refractive index of the portion of the object with respect to d Line.

4. The condensing objective optical system according to claim 1, wherein
a portion, of the photoacoustic element, through which the optical wave passes satisfies Expression (6):
$$1.729 \leq np \leq 2.051 \qquad ( 6 )$$
where
np is a refractive index of the portion of the photoacoustic element with respect to d Line.

5. The condensing objective optical system according to claim 1, wherein the photoacoustic element has a concave face portion facing the object.

6. A photoacoustic device comprising the condensing the condensing objective optical system according to claim 1, wherein the photoacoustic device detects a photoacoustic wave emitted from the object that absorbed an optical wave emitted from the condensing objective optical system.

7. The photoacoustic device according to claim 6, further comprising a light source that outputs two or more kinds of optical waves having different wavelengths to the condensing objective optical system.
